(19) United States
(12) Patent Application Publication
(10) Pub. No.: US 2008/0075777 A1
(43) Pub. Date: Mar. 27, 2008

(54) APPARATUS AND METHODS FOR PREPARING SOLID PARTICLES

(76) Inventor: Michael T. Kennedy, Newbury Park, CA (US)

Correspondence Address: BELL, BOYD & LLOYD, LLP, P.O. Box 1135, CHICAGO, IL, 60690, US

(21) Appl. No.: 11/831,661

(22) Filed: Jul. 31, 2007

Related U.S. Application Data
(60) Provisional application No. 60/820,917, filed on Jul. 31, 2006.

(51) Int. Cl. A61K 9/14; A61K 9/10; B01D 9/02

(52) U.S. Cl. 424/484; 422/245.1; 424/489

(57) ABSTRACT

New processes and apparatuses are disclosed that can be used to form particles from a wide variety of particle-forming materials or mixtures of particle-forming materials contained in solutions, including dissolved, dispersed, suspended or emulsified solutions. To this end an apparatus is disclosed that includes an enclosed spray chamber having a spray nozzle and a collection reservoir for collecting droplets or particles. In an embodiment the spray chamber can include a gas inlet and a gas outlet and the spray nozzle can be located in a gas layer above the level of the gas outlet and the collection reservoir can be located below the level of the gas outlet. The gas outlet can lead to a vacuum source such that a vacuum, can be applied to the container to remove gas from its interior.

### BACKGROUND

Small particles containing a mixture of chemical components are widely useful in a variety of industries. For example, in the pharmaceutical industry small particles can be used to form active pharmaceutical ingredients (APIs) that can be included in a wide variety of dosage forms. Alternatively, the particles may be the basis for the formulation itself, such as in particles manufactured for sustained or controlled release formulations or for pulmonary delivery formulations. In a broad sense, any industry producing dry powdered material as an end-product or intermediate could potentially be served by new apparatuses and methods for producing them.

With respect to pharmaceutical applications, small particles find use, among other things, in controlled release formulations. Controlled release formulations were developed in response to the need to treat illnesses or conditions with a constant level of medicaments over sustained periods of time to provide the more effective prophylactic, therapeutic or diagnostic results. In traditional formulations medicaments were given in doses and at intervals that resulted in fluctuating medication levels. Small particles and microparticles have the capacity to control and stabilize the level of medication delivery by encapsulating an API or medicament inside certain biodegradable materials. Microparticles for systemic delivery of medicaments have the benefit of being injectable, generally with a small gauge needle, either sub-cutaneously or intramuscularly, without the need for incision or implantation. Once administered, the inherent biodegradability of certain polymers used in such compositions improves or modulates the release of the medicament and provides for an evenly controlled level of medication.

Various methods are known for creating dry small particles, such as microparticles, from solutions containing dissolved, dispersed or emulsified components. These methods include spray drying, freeze-drying and spray freeze-drying, solvent extraction, precipitation, phase separation (coacervation), and solvent evaporation or sublimation. In addition, a spray freeze technique to form frozen particles, droplets and/or microdroplets may be combined with solvent extraction. Spray-freezing can be used to avoid high temperatures that could degrade sensitive compounds and can often be performed in a cryogenic solvent so as to provide, under the appropriate conditions, slower mass transport resulting in high encapsulation efficiency and the preservation of the solution microstructure within nascent particles. Once droplets are frozen, solvent extraction can be used as an alternative to freeze drying (sublimation and evaporation), and can be used to produce alternative particle morphologies and more efficient removal of some solvents.

Processes for freezing droplets containing drug formulations with dissolved or dispersed components and removing solvent to produce dry particles have been described. U.S. Pat. No. 3,928,566 (Briggs, et al.) describes a process for preparing homogenous solid particulate blends by spraying a solution or colloid suspension containing a biologically active component into a fluorocarbon refrigerant and lyophilizing the resulting frozen droplets. The solution or colloidal suspension is said to be sprayed into a moving bath of boiling fluorocarbon refrigerant below −20° C. to freeze the droplets, after which the frozen droplets are subjected to vacuum while maintaining vacuum at a suitable temperature to sublime the solvent and form particles. Similarly, U.S. Pat. No. 4,704,873 (Imaike, et al.) describes a method for producing microfine frozen particles by atomizing liquid with a two fluid nozzle and directing the spray into a refrigerant liquid (liquid nitrogen, cooled organic solvent, below −20° C.) whose surface is said to be stirred by application of kinetic energy to form ripples at the freezing surface which is said to help prevent particles from agglomerating prior to freezing. U.S. Pat. No. 4,848,094 (Davis, et al.) describes a method and apparatus for producing frozen spherical droplets by feeding a liquid of organic or biological material through a nozzle as a continuous stream. After allowing the liquid to break up into droplets by traveling through a gas or vapor, the droplets are said to be frozen in a cryogenic liquid and then separated from the liquid in the apparatus. However, extraction, lyophilization or vacuum drying of the droplets to produce dry particles is not described, and the gaseous phase merely provides the distance sufficient to cause the stream to break apart before contacting the cryogenic liquid. Each of the above methods requires maintenance of the droplets below the freezing point of their solvent for vacuum drying.

Similarly, techniques for encapsulating active agents into polymer beads, microspheres, or microparticles have been described. U.S. Pat. No. 4,272,398 (Jaffe, 1981) describes a solvent evaporation method that is said to encapsulate pesticides in bioerodable polymers by co-dissolving the polymer and substance in organic solvent, dispersing the solution in an aqueous medium, and evaporating the solvent. U.S. Pat. No. 4,166,800 (Fong, 1979) describes a method for forming microspheres, either as microcapsules, or as microprills, which are homogenous mixtures of a core material (polymer) and drug, by a phase separation technique. Microprills are said to be made by dissolving the polymer and drug in a solvent and lowering the temperature of the solution to 40 to −100° C. and adding a polymer-drug non-solvent to form the discrete microprills by precipitation. Cold temperatures are said to help stabilize the particles during phase separation. U.S. Pat. No. 5,342,557 (Kennedy, 1994) describes a method for producing microparticles by melting a polymer, such as PLGA, and extruding the melt through a capillary, spray nozzle, or rotary atomizer at a temperature and viscosity that minimizes formation of fibers. Particles are said to form as they cool by freely falling in air or by introduction into a cooling liquid such as liquid nitrogen or pentane. The range of polymers having suitable viscosities limits the usefulness of this technique because many biological agents cannot be incorporated into such particles due to their inability to be dispersed and sensitivity to thermal degradation among other physical limitations.

U.S. Pat. No. 5,102,983 (Kennedy, 1992) describes a process for forming foamed bioabsorbable polymer microparticles using a spray-freeze drying technique. A solution of dissolved polymer is said to be introduced in small discrete quantities into a liquid, such as chilled pentane, which is immiscible with the solvent and which is said to freeze the polymer solution into particles. The solvent is said to be removed from the frozen polymer particles under vacuum to provide the foamed particle. Temperature differences of at least 10° C. between the freezing temperature and the polymer solvent freezing point are noted, and the drying step is said to be conducted at or below the melting point of the frozen polymer particles to maintain the particles in the frozen state before and during the vacuum solvent removal operation. Residual solvents may be removed by warming the particles later. The method is limited to the production of porous particles.

U.S. Pat. No. 5,019,400 (Gombotz, et al., 1991) describes a method for preparing microspheres using very cold temperatures to freeze polymer-biologically active agent mixtures. Single-phase solutions or suspensions are said to be atomized through ambient open air, rather than in an enclosed system, into a vessel containing a liquid non-solvent, alone or frozen and having a liquefied gas over-lay, at a temperature below the freezing point of the polymer/active agent solution. Droplets are said to freeze on contact with the cold liquefied gas or non-solvent, and as the droplets and non-solvent for the polymer are warmed, the solvent in the droplets thaws and is said to be extracted into the non-solvent, resulting in hardened microspheres. The use of a liquefied gas overlay of a frozen layer is not practical for scale up, and the lack of mixing during the thawing processes leads to inefficient extraction and undesirable temperature and concentration gradients. The lack of control over the temperature profile through time during the thawing process can also cause undesirable effects such as porous or hollow particles, internal phase separation with co-solvent systems or emulsions and excessive uptake of the non-solvent into the polymer matrix.

U.S. Pat. No. 6,726,860 (Herbert et al.) and related applications U.S. Pat. Nos. 6,358,443; 6,153,129; 5,922,253 describe an apparatus and method for producing microparticles from a solution containing a biocompatible polymer, solvent, biological agent and other excipients by spray freezing, followed by extraction of the polymer solvent by a non-solvent that is in the liquid state throughout the method. Freezing is said to occur in a freezing section or freezing vessel which is encircled by a radially dispersed liquefied gas. The technique is limited by the difficulty in obtaining suitable radial displacement of the liquefied gas directing means, which must operate efficiently to avoid freezing of the atomization nozzle (Col 3, lines 49-58). Extraction is said to occur in an extraction section or separate extraction vessel containing the liquid non-solvent for the polymer. Frozen droplets, liquefied gas, and cold gas are said to pass together through a three-phase communication port separating the sections/vessels. The device is a complex multivessel device in which freezing and extraction occur in separate vessels. The nozzle is located in close proximity to the liquefied gas and the particle collection process is initiated in close proximity to the nozzle. This can cause polymer precipitation and freezing in and around the nozzle. Such freezing can interfere with the operation of the nozzle. In addition, the close proximity of the nozzle to the liquefied gas limits the opportunity for solvent to evaporate from the particle after the spray droplet forms.

Additional spray-freeze techniques have been devised for making microparticles containing biologically active agents for non-parenteral applications. U.S. Pat. No. 6,103,269 (Wunderlich, et al) describe a method and apparatus for making round granules or pellets containing hydrophilic macromolecules for pharmaceutical purposes, primarily for tableting, gel caps, or rapid release oral delivery. A structural agent (hydrophilic macromolecule) is said to be dissolved in a solvent, the active agent dispersed in the solution and the mixture added drop-wise, rather than through a spray nozzle, to a deep-cooled liquefied gas (liquid nitrogen) to form a solid (inert liquid). The frozen particles are removed from the cryogenic bath continuously using a conveyor system and are transferred to a freeze-dryer for water removal. U.S. Pat. No. 6,753,014 (Sjoblom, 2004) describes a general method, but not an apparatus, for preparing homogenous microparticles containing a pharmaceutically active substance by spray-freezing a solution, suspension or emulsion (containing a polymer binder) into a cold boiling liquefied gas and sublimating the frozen droplets to remove liquid in a conventional freeze-dryer. In particular, ‘high dry content, low friability’ particles with drug contents of greater than 50% by mass are said to be produced. The particles are said to be useful for spray-coating in a fluid-bed coater. The author describes the single drying step, high active content, and ability to form non-porous particles as advantageous over the method of U.S. Pat. No. 5,019,400 for producing particles. In addition, U.S. Pat. No. 6,862,890 (Williams III, et al.) describes an apparatus and method for spray freezing into a liquid to form 10 nm to 10 μm diameter particles. An insulating nozzle in the system is specifically located at or below the surface of the cryogenic liquid, rather than in a temperature layer or zone above the collection reservoir. Collected particles are removed and dried by other means such as lyophilization or a cryogenic atmospheric fluidized bed apparatus. Smaller particles necessarily result from the high pressures and velocities associated with the nozzle to enable it to function so closely or beneath the cryogenic liquid. Specialized nozzles are also required for this operation.

U.S. Pat. No. 7,007,406 (Wang et al.) describes spraying a carrier liquid containing a powder forming ingredient to form a flow of liquid droplets; entraining the flow in a concurrent coolant flow for sufficient time to freeze the liquid droplets into frozen particles; collecting the particles on a frit at the bottom of the spray chamber and drying the frozen particles. The nozzle is located in close proximity to the liquefied gas, which can cause polymer precipitation and freezing in and around the nozzle. Such freezing can interfere with the operation of the nozzle. In addition, the close proximity of the nozzle to the liquefied gas limits the opportunity for solvent to evaporate from the particle after the spray droplet forms.

### SUMMARY

New processes and apparatuses are disclosed that can be used to form particles from a wide variety of particle-forming materials or mixtures of particle-forming materials contained in solutions, including dissolved, dispersed, suspended or emulsified solutions. To this end an apparatus is disclosed that includes an enclosed spray chamber having a spray nozzle and a collection reservoir for collecting droplets or particles. In an embodiment, the spray chamber can include a gas inlet and a gas outlet and the spray nozzle can be located in a gas layer above the level of the gas outlet and the collection reservoir can be located below the level of the gas outlet. In this embodiment, the gas outlet can lead to a vacuum source such that a vacuum, can be applied to the container to remove gas from its interior.

In an embodiment, the gas inlet can be above the level of the gas outlet. Generally, the gas inlet above the level of the outlet can be used to introduce a gas that is suitable to prevent or avoid nozzle fouling, for example, due to clogging or freezing during the spray process. In an embodiment, the apparatus can include a second gas inlet below the gas outlet which can be used to introduce a gas suitable for freezing or chilling the spray droplets as they pass through a lower gas layer.

In an embodiment, the collection reservoir can contain a collection fluid which can be a liquefied gas. In an embodiment, the collection fluid can be an anti-solvent that is suitable for extracting solvents from nascent frozen particles. In an embodiment, the collection fluid can also contain particles.

In an embodiment, a feed tube can extend into the spray chamber which can be used to add collection fluid when the levels of collection fluid drop due to evaporation, boil off or transfer to downstream operations. The feed tube can extend into the collection reservoir so that when the collection fluid is added to the collection reservoir, it will not disrupt the gas layer or create currents in the enclosed container.

In an embodiment, the apparatus can be used to generate and maintain at least two gas layers which can have distinct temperature profiles. Generally, the gas layers are arranged one above the other in the enclosed chamber. The border between the gas layers can be created by the outlet port through which gases from both layers can escape or alternatively, be withdrawn through a vacuum. In an embodiment, the apparatus can be configured such that a warmer gas above the freezing or precipitation points of the particle-forming mixture is introduced through the inlet port to form a warm gas zone. In an embodiment, a cold gas can be formed by evaporation of a liquid gas in the collection reservoir or by introducing a cold gas through one or more gas inlets that are below the outlet. These alternatives are compatible and in an embodiment can be used together. Thus, in an embodiment, the enclosure can have two gas layers in which a relatively warmer gas layer is generally positioned above a relatively colder gas layer in an enclosed container.

Such apparatuses can be used to prepare solid particles such as microparticles. In a method, a first gaseous temperature zone having a temperature above the freezing or precipitation points of a particle forming solution is formed in the enclosure. Then a pool of a cold collection fluid is introduced into the closed container in the collection receptacle. A mixture of a particle-forming material in a suitable liquid is prepared and the mixture is sprayed through the first gaseous temperature zone directly into the pool of collection fluid to form a frozen particle.

In a method, a second gaseous temperature zone below the first gaseous temperature zone can be used to freeze or chill the droplets before they enter the collection fluid in the collection reservoir.

In a method, the nascent frozen particles are extracted with an anti-solvent to remove at least a portion of the solvent from the frozen particles. This can be carried out at a temperature below the freezing or precipitation point of the particles. In one method, the steps of freezing and extracting the particles is carried out in a single apparatus. In a method, the steps of freezing, extracting and drying are carried out in a single apparatus. In an additional method, frozen particles can be transferred either in a continuous or batch manner to additional pieces of equipment for extraction and/or drying.

In a method active agents can also be included in the mixture of particle forming material and incorporated in to particles. Alternatively, active agents can be added to particles after they are formed.

The disclosed apparatuses and methods can be used to form particles having a wide range of morphologies, including hollow, porous or solid particle structures. Such particles can find use in many pharmaceutical formulations, including sustained release, parenteral and oral pharmaceutical dosage forms. They will be suitable for delivery through pulmonary, buccal or other routes. In addition, the particles are suitable for use in a wide range of consumer product applications.

Apparatuses and methods can be used to produce microparticles at high yield with control over size. Apparatuses for carrying out the process are suitable for laboratory use and pilot/commercial scale operations and are generally enclosed to provide for containment of potent compounds, aseptic processing and process control reproducibility. The device can use common commercial atomizing spray equipment and liquefied gases or other cryogenic fluids with minimal clogging or freezing. The apparatus facilitates rapid removal of solvent from nascent particles and provides for control over the temperature conditions throughout the process to allow for engineering of various particle morphologies.

To this end, a method and apparatus is disclosed for spray freezing solutions to form small solid particles, such as microparticles. The particles can then be freeze-dried or extracted with a cold non-solvent to remove solvent from the particle forming material leaving a stable solid particle. In an embodiment, the device is an enclosed system and can have multilayered temperature zones in a gas phase. A spray atomization nozzle can be located in a relatively warmer gas zone, which can have a temperature above the melting/precipitation temperature of the particle-forming material and its solvent. The warmer gas zone prevents precipitation or solid formation and clogging of the spray atomization nozzle. The relatively warmer gas zone also facilitates evaporation of solvent from the spray droplets as they pass through the zone. Spray droplets can be formed at the nozzle and can pass through the relatively warmer gas zone into a relatively colder gas zone which contains a sufficiently cold gas to prechill or even freeze the particles as they pass through into a collection fluid in a collection reservoir. While particles are being collected in the collection fluid, the collection fluid has a temperature that is sufficiently cold to keep the particle as a frozen solid.

In an embodiment, the device can be configured to have at least two layers wherein the temperature profile in each layer can be defined and the temperature profile across the transition between the two gas layers may have a nonlinear or discontinuous or steep temperature transition between two gas layers. The apparatus allows particles to be rapidly cooled or frozen with minimal precipitation or phase separation.

Additional features are described herein, and will be apparent from, the following Detailed Description and the figures.

### BRIEF DESCRIPTION OF THE FIGURES

FIG. 1 illustrates an exemplary enclosed spray chamber.

FIG. 2 illustrates an exemplary temperature profile that can be produced by an embodiment of the device.

FIG. 3A illustrates an exemplary spray chamber.

FIG. 3B provides a graph showing the variation in temperature between the nozzle tip and liquid surface.

FIG. 4 illustrates an exemplary spray chamber.

FIG. 5A provides a scanning electron micrograph of particles prepared according to Example 3.

FIG. 5B provides a scanning electron micrograph of the cross section of a particle prepared according to Example 3.

FIG. 6A provides a scanning electron micrograph of particles prepared according to Example 4.

FIG. 6B provides a scanning electron micrograph of the cross section of a particle prepared according to Example 4.

FIG. 7A provides a scanning electron micrograph of particles prepared according to Example 5.

FIG. 7B provides a scanning electron micrograph of the cross section of a particle prepared according to Example 5.

FIG. 8A provides a scanning electron micrograph of particles prepared according to Example 6.

FIG. 8B provides a scanning electron micrograph of the cross section of a particle prepared according to Example 6.

FIG. 9A provides a scanning electron micrograph of particles prepared according to Example 7.

FIG. 9B provides a scanning electron micrograph of the cross section of a particle prepared according to Example 7.

FIG. 10A provides a scanning electron micrograph of particles prepared according to Example 8.

FIG. 10B provides a scanning electron micrograph of the cross section of particles prepared according to Example 8.

FIG. 11A provides a scanning electron micrograph of particles prepared according to Example 9.

FIG. 11B provides a scanning electron micrograph of the cross section of particles prepared according to Example 9.

FIG. 12A provides a scanning electron micrograph of particles prepared according to Example 10.

FIG. 12B provides a detailed scanning electron micrograph of particles prepared according to Example 10.

FIG. 12C provides a scanning electron micrograph of the cross section of particles prepared according to Example 10.

FIG. 12D provides plot of X-ray powder diffraction measurements for an amorphous solid dispersion of 15 wt % N-(4-(6-(4-(trifluoromethyl)phenyl)pyrimidin-4-yloxy)benzo[d]thiazol-2-yl)acetamide in a polymer, HPMCAS, made by spray freeze/solvent extraction, according to Example 10

FIG. 13 provides a graphical representation of temperature profiles for the headspace for ALN2 and ACES process vessels. A indicates cryogen level in ACES, and the vacuum port level in ALN2 (which delineate the top of a cold vapor zone). B indicates the cryogen level in ALN2.

FIG. 14 is graphical representation of the relationship between the size of a particle in a vessel, its calculated residence time (dash lines) in the headspace, and its calculated freezing time (solid lines) in the ALN2 process. Dichloroethane (solid symbols) and Dichloromethane (open symbols). Calculated t_{f}>tᵣₑₛ, noted by *(dichloromethane) and ≠(dichloroethane), indicate incomplete freezing prior to impact with the cryogen bed.

FIG. 15 provides a graphical representation of modeled particle temperature (dashed lines) and vessel temperature (solid line) as a function of temperature through the headspace in the ALN2 process. Calculated temperature is for median-sized droplet of dichloromethane (dash line) or dichloroethane (dash-dot line). A and B indicate completion of dichloroethane and dichloromethane droplet freezing, respectively. X-axis depicts distance from nozzle tip.

FIG. 16 provides scanning electron micrograph pictures of particles made according to Example 11 with the ALN2 process. A-C are for particles prepared in dichloromethane and D-F are for particles prepared in dichloroethane.

FIG. 17 provides scanning electron micrographs of the structure of PLGA microparticles prepared by ACES conditions with long freezing times, using dichloromethane (FIGS. 17 A-C) and dichloroethane (FIGS. 17 D-E) according to Example 11.

FIG. 18 provides scanning electron micrographs of the structure of PLGA microparticles prepared by ACES conditions with intermediate freezing times, using dichloromethane (Fig. A-C) and dichloroethane (FIGS. 18 D-E) according to Example 11.

FIG. 19 provides scanning electron micrographs of the structure of PLGA microparticles prepared by ACES conditions with rapid freezing times, using dichloroethane and pentane (FIGS. 19 A-C) and dichloroethane and iso-pentane (FIGS. 19 D-E) according to Example 11.

FIG. 20 illustrates an embodiment of the device in which multiple spray chambers are assembled into a system.

### DETAILED DESCRIPTION

Generally, an apparatus includes an enclosed spray chamber having a spray nozzle and a collection reservoir for collecting droplets or particles. The spray chamber can have one or more gas inlets and one or more gas outlets and the spray nozzle can be located in a gas layer above the level of the gas outlet and the collection reservoir can be located below the level of the gas outlet.

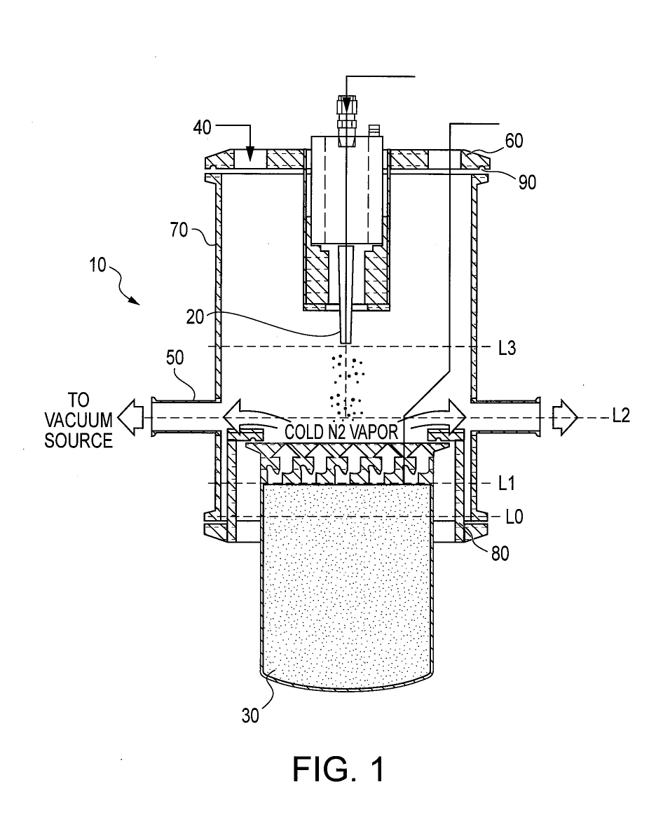

An embodiment of an apparatus is illustrated in FIG. 1 and includes an enclosed spray chamber 10 containing the following components:

(1) a spray nozzle 20 that is in fluid communication with a source that contains a mixture of a particle-forming material and a liquid for feeding a dissolved, emulsified or suspended particle-forming spray through the chamber 10;

(2) a collection reservoir 30 at the bottom of the chamber for holding the collection fluid and collecting the spray droplets or particles of particle-forming material and maintaining the droplets in a frozen state;

(3) 1 or more inlet ports 40 that allow a gas having a first temperature to enter through a top portion of the chamber;

(4) 1 or more outlet ports 50 which can vent both a lower gas having a second temperature over the collection fluid and also the upper gas phase in the vicinity of the spray nozzle, such that a relatively sharp temperature transition is created between the upper and lower gas phases at the approximate level of the port(s). The port(s) can be connected to a vacuum source to control the rate of gas removal and facilitate the formation of this temperature transition zone.

Optionally, collection reservoir 30 can be a vessel which can be moved to another location after formation of the particles. At the other location, further processing can occur, for example cold collection fluid can be exchanged with anti-solvent that extracts solvent from the nascent particles. This allows particle formation to continue in a relatively continuous manner, being interrupted only to change out the collection reservoirs 30.

The spray chamber 10, gas inlet 40 and gas outlet 50 ports can be made of any material that can withstand conditions during sanitization and steam sterilization of the inside of chamber 10, and can also withstand the temperatures and gas pressures used to form microparticles. In addition, the materials should generally be nonreactive with the particle-forming solutions and any active agents or gases that are passed through the system. It is not necessary that all parts of the apparatus be made of the same material, for example the spray chamber could be made of stainless steal and the spray nozzle made from another metal alloy. Suitable materials include stainless steel, metal alloys, plastics, elastomers, glass and the like.

Any suitable number of gas inlet 40 ports can be included in spray chamber 10. Although for many purposes one port will be sufficient, two, three or four or more ports could be included. Additional ports could be used to maintain or control the temperature profile around the circumference of the warmer gas chamber or control air currents, as desired. Metering and control valves can be installed within any of these ports to provide for controlled gas or liquid flows to and from the chamber.

In an embodiment, the gas inlet 40 can be located above the level of the gas outlet such that gas can enter through the inlet port to keep the spray nozzle insulated from colder gas temperatures in proximity to the collection fluid, which is generally cold enough to freeze the droplets. Gas can be passed through a filter and into the chamber through the inlet 40 and can then flow through the chamber and exit through a gas outlet port 50. The inlet gas can be any gas that is not reactive with the spray nozzle or the particle forming solutions sprayed through the spray nozzle. Suitable gases can include ambient air, inert gases, CO₂, N₂, controlled air, or their mixtures, for example. The gas is generally warm enough to avoid or minimize precipitation or freezing of components of the particle-forming material.

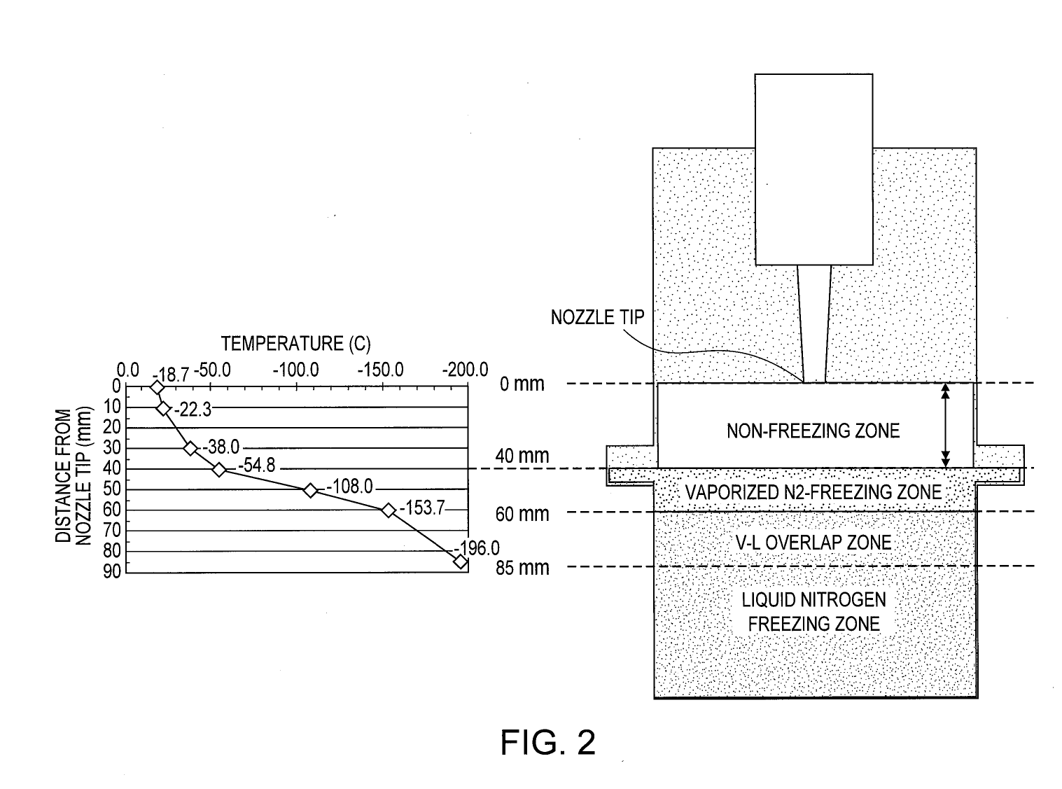

In an embodiment, the temperature of the inlet gas can be controlled such that the gas layer above the gas outlet has a desirable temperature profile. For example, as shown in FIG. 2, a relatively warm inlet gas, which can be at ambient temperature, enters the chamber through the inlet and gradually cools to about −20° C. as it passes the nozzle tip, down through the chamber toward the cryogenic collection fluid below. As the gas traverses the chamber to the level of the outlet port 50, it gradually cools to about −55° C. The relative warmth at the level of the spray nozzle prevents freezing and precipitation of particle-forming material on the nozzle during droplet formation such that the system can be kept in operation for lengthy periods of time without the need to stop the process to clean a fouled nozzle. In addition, the relatively warm gas layer allows a portion of the solvent from within the spray droplets to evaporate as the droplets pass through the warm gas zone. The temperatures chosen will depend upon the nature of the liquid and the other components of the particle-forming mixture. Cooler gases are available when the materials in the particle-forming mixture are less likely to precipitate or freeze.

In an embodiment, the walls of the chamber above gas outlet 50 can be heated to warm the gas in this zone.

As illustrated in more detail in the embodiment of FIG. 1, gas inlet port 40 can be mounted on a lid 60 that covers and is sealed to spray chamber 10 through a seal 90. However, gas inlet(s) 40 can be positioned at any location that allows them to be used to maintain the temperature of the spray nozzle above the freezing and precipitation point of the particle-forming mixture. Inlets could be located on the walls 70 of the chamber at any height that can be used to keep the spray nozzle relatively warm and clear during use.

As illustrated in the embodiment of FIG. 1, the spray chamber is covered by a lid which is joined to the chamber through a seal 90. Other configurations in which the top is manufactured in one piece with the side walls of the chamber are also envisioned and can be used. In such a configuration, the spray atomizer 20 and gas inlet port 40 could be mounted to the spray chamber 10 in a similar or identical manner.

Any spray atomizer that can produce droplets from the liquids of the present invention can be used. Suitable spray atomizers include two-fluid nozzles, single fluid nozzles, ultrasonic nozzles such as the Sono-Tek™ ultrasonic nozzle, rotary atomizers or vibrating orifice aerosol generators (VOAG), and the like. Atomizers can be connected to spray chamber 10 by any method known in the art. In an embodiment, the connection allows for atomizer 20 to be raised and lowered in the chamber.

The embodiment of FIG. 1 also shows gas outlet port 50 which can be located above collection reservoir 30 and below the atomization nozzle 20 and inlet port 40. Optionally, outlet port 50 can be connected to a vacuum line to control the amount of gas escaping the chamber and to control the temperature profile within the chamber. Metering and control valves can be installed within this line to control the flow out of the chamber.

The collection reservoir can be joined to the spray chamber by any suitable method. For example, the connection can be through an adapter or a flange which can be either removable or permanent. In addition, the collection reservoir can be an integral part of the enclosure. In the embodiment illustrated in FIG. 1, a collection reservoir 30 is mounted below the gas outlet port 50 on adapter 80. Collection reservoir 30 can be made of any material that can withstand the cold temperature of cryogenic liquids and that is stable to solvents and other particle-forming materials used in the particle preparation process. Collection reservoir 30 and adapter 80 can be conveniently mounted to chamber 10 and sealed through the use of gaskets at their connection points. This enables disassembly of the chamber for cleaning, and in the illustrated embodiment, removal of the collection reservoir 30 following particle collection for downstream particle processing.

In alternate embodiments, the collection reservoir can be permanently affixed to the spray chamber vessel for carrying out downstream particle processing steps without changing out the collection vessels. In an embodiment, the collection reservoir can be insulated to limit heat gain or equipped with internal or external heat transfer surfaces to modulate temperature. In another embodiment the frozen particles can be continuously siphoned off or otherwise removed through a port located within the collection reservoir and collected for subsequent processing.

In operation, collection reservoir 30 is filled to a suitable level with a cryogenic collection fluid. Suitable collection fluids include liquids that will hold newly formed or nascent frozen microparticles as a solid for further processing. Suitable collection fluids include liquefied gases and anti-solvents, as will be described in more detail below. The level of the collection fluid can be varied within the collection reservoir such that on the low end there is sufficient fluid to collect and hold the particles and, on the high end, to the vicinity of the top of the reservoir.

To maintain the level of the collection fluid when highly volatile liquefied gases are used, a collection fluid feed tube can be positioned in the spray chamber with an end extending into or above the collection reservoir. A level monitoring device such as a thermocouple probe can be positioned at the desired collection fluid level and connected to a controller that controls a valve that regulates the flow of liquefied gas into the feed tube to refill the collection reservoir 30. Thus, when the level of the collection fluid drops below the thermocouple, the temperature detected by the thermocouple will rise substantially above the temperature of the collection fluid to provide a signal that additional collection fluid can be added to the collection reservoir 30.

In one embodiment, a temperature controller responds to the thermocouple probe temperature at the desired collection fluid level. With liquid nitrogen the temperature set point of the controller can be set at or above the liquid's boiling point temperature, and when the level of liquid nitrogen collection fluid is below the thermocouple, the temperature will rise above this temperature and signal the valve to open. Liquid nitrogen will then flow into the reservoir until it reaches the thermocouple sending its temperature reading to below the set point and signaling the liquid nitrogen flow to stop. This configuration provides for the introduction of cold liquids into the collection reservoir to maintain a desired collection fluid level and avoids disruption of the gas temperature zones above the liquid level or requiring spray atomization to be halted.

In operation the device can have three distinct temperature zones. The collection fluid generally provides the coldest temperature zone in which liquid spray droplets can be frozen and maintained in the frozen state until downstream processing. There is also a gas zone between level L1 and L2 (FIG. 1) which is generally a colder gas zone in which liquid spray droplets are chilled or freeze as they pass. Above L2 is a third temperature which is of a suitable temperature to prevent precipitation or freezing around the spray atomization nozzle during the spray operation.

In an embodiment of the apparatus, the size of each layer or temperature zone can be modified. For example, with reference to FIG. 1, the position of the atomization nozzle can be adjusted upward or downward to alter the axial location of plane L3, thus affecting the distance from L2 to L3 corresponding to the distance that spray droplets travel through the warm gas zone. The level control of the liquid at L1 may be altered to a lower plane, such as L0, in a similar manner to change the depth of the cold vapor layer. This can be accomplished by raising or lowering the level monitoring device that regulates the flow of collection fluid into the collection reservoir.

Additional operations can be used to control the temperature gradients in each zone. For example, control of the vacuum can alter gas boil-up rate, when liquefied gases are used as collection fluids, thus changing the cold vapor zone temperature gradient; alternatively, heat can be applied to the liquefied gas to enhance its boil-up rate. For example, with reference to FIG. 1, Increasing inlet air flow or temperature through gas inlet port 40 can be used to alter the profile of the temperature zone above L2. Additional heating or cooling mechanisms can be applied to either of the three zones by any of a variety of methods known in the art to generate a desired temperature profile in each zone. The temperature gradient within the chamber can easily be determined by placing multiple temperature monitoring devices, such as thermocouples, within the chamber at desired heights or by raising or lowering a single temperature monitoring device while measuring the temperature as a function of position of the device within the chamber under a given set of conditions.

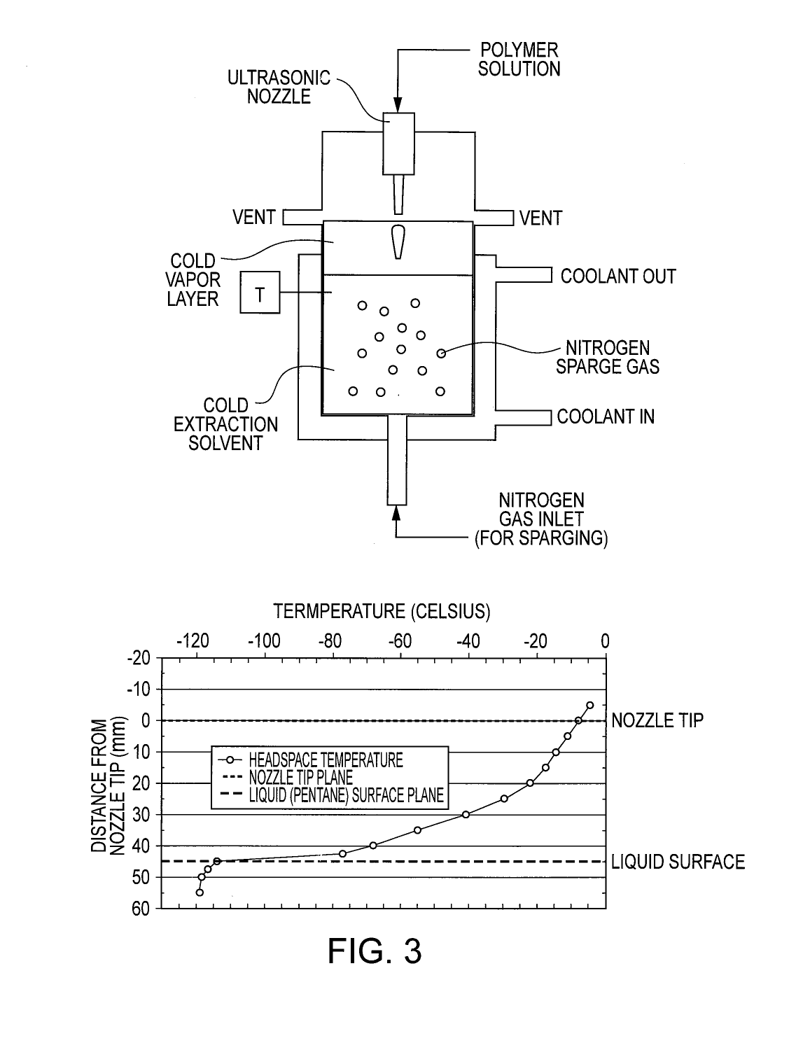

With reference to FIG. 1, the gas layer between L1 and L2 can be generated in any suitable manner. For example, when liquefied gases are used as the collection fluid in the collection receptacle, they can boil off to generate the cold gas temperature zone below outlet port 50. Alternatively, the cold gas layer can be created by adding an inlet port for cold gas between the level of the collection fluid and the outlet port. In yet another embodiment, a cold gas can be bubbled through the collection fluid in the collection reservoir as through a sparger or through an inlet port covered by a frit such as a stainless steel frit (FIG. 3). The later methods may be particularly suitable to methods that utilize cold antisolvents, rather than liquefied gases, as collection fluid.

It is within the skill of one having skill in the art to choose suitable materials and make suitable connections to obtain the apparatuses. For example, gas tight seals for inlet and outlet ports and seals between the lid and the side walls of the chamber, and vacuum connections to the outlet port can easily be created.

The apparatus enables safe fabrication of drug-loaded microparticles within a contained environment on a laboratory bench. The apparatus provides for atomization in an enclosed chamber environment using standard atomization nozzles without allowing the spray nozzle to get so cold due to temperature effects from the collection fluid that it becomes clogged from precipitation or freezing of the particle forming solution. Without the proper design of fluid and gas flows, cold vapor produced by the boiling liquid nitrogen would infiltrate the entire chamber, resulting in: (1) premature freezing of the feed solution within the fluid path to the spray nozzle tip, and (2) chilling the electromechanical (ultrasonic) atomizer to a point that was outside its physical operating limitations. The apparatus can provide for consistent nozzle atomization by creation of the multi-layered temperature zone feature in the enclosed chamber.

The present disclosure also contemplates atomization of active agents, including biologically active compounds, within an enclosed chamber, with the added flexibility of enabling easy change out of batch containers to enable production of multiple batches of materials within the lab environment on a given day. The apparatus is well suited for use with active agents, including biologically active compounds that can exert physiologic effects in small quantities. A process such as atomization greatly enhances the risk of exposure by inhalation or contact increasing the need for carrying out such a process in a sealed chamber. However, other processes may benefit from the sealed chamber design including those involving volatile or toxic solvents, or those requiring aseptic sterile processing. The design, with minor modifications, would be expected to work in each of these applications, as the process streams in and out of the unit may all be controlled or filtered to accomplish the objectives of a specific application.

In one embodiment the apparatus provides, in a sealed chamber, a fixed cold vapor layer that does not interfere with nozzle performance. The apparatus provides for adjustment of the relative distance between the nozzle tip and the cold vapor layer, as well as the relative thickness of the cold vapor layer above the liquefied gas or collection fluid.

Generally, the particle preparation process involves dissolving or suspending the particle forming material, such as a polymer or other solid forming agent, in a suitable solvent at the desired concentration. Optionally, an active agent can be included in the particle-forming material liquid mixture and solubilized or suspended along with the particle-forming material. The solution, suspension or emulsion of the particle-forming material can be atomized into a relatively warmer gas phase through which the droplets can pass into a relatively colder gas phase and then into a pool of collection fluid in which the droplets are maintained in a frozen form. During this process the droplets containing the particle-forming material and its solvent are formed and then rapidly frozen. The frozen droplets can be formed either in the collection fluid or in the cold gas phase above the liquid. The collection fluid can be any liquid in which the nascent particles can be formed and/or held as stable solids, as described in more detail below.

The collection fluid can then be exchanged with an anti-solvent that can be used to extract the solvent from the particle. Exchange can be accomplished by allowing the liquid to boil-off (in the case of liquid gases), by filtration or by decanting so that the particles are nearly free of the cryogenic liquefied gas and then adding the anti-solvent. In some circumstances the exchange step may be unnecessary, such as when the particles are collected directly in cold anti-solvent. As solvent is extracted from the nascent microparticles, the particles become stable solids and temperatures can be allowed to rise.

The solvent in the nascent particle can then be extracted into the anti-solvent. The extraction can take place in a temperature controlled system. On the low end, the temperature can be any temperature at which the anti-solvent is a liquid. On the high end, the temperature can be any temperature at which the particles remain in the solid state. The range can be anywhere from about −180 to 20° C., for example, but will ultimately depend on the particle composition, the choice of solvent and anti-solvent, when an anti-solvent is used. In a method, the temperature of the system may be changed over the course of the extraction step, in such a manner that the extraction temperature thermodynamically favors dissolution or miscibility between the solvent and anti-solvent system.

The anti-solvent and extracted solvent can be removed from the particles by any suitable method. For example, temperature controlled filtration can be used such that the particles can be rinsed and recovered by filtration. The recovered particle filter cake can then be dried in a temperature-controlled environment under vacuum or with a forced gas stream to remove residual solvents.

Microparticle preparation begins with preparation of the feed solution. The process can involve dissolving, suspending or forming an emulsion of the particle forming material, such as a polymer or other solid forming agent, in a suitable solvent at the desired concentration. An active agent can also be included in this solution. Once the solution, suspension or emulsion has been prepared, atomization may begin.

A wide range of particle-forming materials can be used in the disclosed system. In fact, any material or mixture of materials that can be dissolved, suspended and/or emulsified and then form a solid particle when passed through spray atomization equipment into a collection fluid of the invention can find use in the present system. Thus, suitable ingredients to particle-forming mixtures include, for example, active agents, such as antioxidants, absorption enhancers, buffers, nucleic acids, peptides, polypeptides, polymers, protease inhibitors, proteins, stabilizers, surfactants, and small molecule drugs and pro-drugs; fillers, plasticizers, and pharmaceutically acceptable carriers, excipients, or stabilizers such as those described in Remington's Pharmaceutical Sciences, 16^{th} Edition, Osol, A. Ed. (1980), provided that they do not adversely affect the desired characteristics of the desired particle or its formation.

Suitable particle-forming materials can include cellulose derivatives, like ethylcellulose, hydroxypropyl methyl cellulose, hydroxypropyl methyl cellulose acetate succinate, hydroxyethyl cellulose, hydroxypropyl cellulose, ethyl hydroxyethyl cellulose, carboxymethyl cellulose, cellulose acetate butyrate, cellulose acetate phthalate, methylcellulose, or polysaccharides, like alginate; xanthan; carrageenan; scleroglucan; pullulan; dextran; hyaluronic acid; chitin; chitosan; starch; etc other natural polymers, like proteins (e.g. albumin, gelatin, etc); natural rubber; gum arabic; etc synthetic polymers, like acrylates (e.g., polymethacrylate, poly(hydroxy ethyl methacrylate), poly(methyl methacrylate), poly(hydroxy ethyl methacrylate-co methyl methacrylate), Carbopol™ 934, etc.); polyanhydrides (e.g. poly(bis carboxyphenoxy)methane, etc.); PEO-PPO block-co-polymers (e.g. poloxamers, etc); polyvinyl chloride; polyvinyl pyrrolidone; polyvinyl acetate; polyvinyl alcohol; polyethylene, polyethylene glycols or co-polymers thereof; polyethylene oxides or co-polymers thereof; polypropylene or co-polymers thereof; polyesters (e.g. poly(lactic acid), poly(glycolic acid), poly(caprolactone), etc., or co-polymers thereof, or poly(ortho esters), or co-polymers thereof); polycarbonate; cellophane; silicones (e.g. poly (dimethylsiloxane), etc); synthetic rubbers (e.g. styrene butadiene rubber, isopropene rubber, etc.); etc. surfactants, e.g. anionic, like sulphated fatty alcohols (e.g. sodium dodecyl sulphate), sulphated polyoxyethylated alcohols or sulphated oils, etc.; cationic, like quaternary ammonium or pyridinium cationic surfactants, etc; non-ionic, like polysorbates (e.g. Tween), sorbitan esters (e.g. Span), polyoxyethylated linear fatty alcohols (e.g. Brij), polyoxyethylated castor oil (e.g. Cremophor), polyoxyethylated stearic acid (e.g. Myrj), etc. other substances, like shellacs; waxes (e.g. carnauba wax, beeswax, glycowax, castor wax, etc); nylon; stearates (e.g. glycerol palmitostearate, glyceryl monostearate, glyceryl tristearate, stearyl alcohol, etc.); lipids (e.g. glycerides, phospholipids, etc); paraffin; lignosulphonates; etc.

Many types of polymer can be used, provided the appropriate solvent or non-solvent mixture are found having suitable melting temperatures. In general, a polymer solution can be prepared with about 1% polymer to about 20% polymer, or about 5-10% polymer by mass. Suitable polymers include bioerodible polymers such as poly(lactic acid), poly(lactic-co-glycolic acid), poly(caprolactone), polycarbonates, polyamides (e g polyacrylamide, poly(methylene bisacrylamide), etc), polyanhydrides, polyamino acids, polyortho esters, polyacetals, polycyanoacrylates or degradable polyurethanes, or non-erodible polymers such as polyacrylates, ethylene-vinyl acetate copolymers or other acyl substituted cellulose acetates or derivatives thereof, non-erodible polyurethanes, polystyrenes, polyvinyl chloride, polyvinyl fluoride, poly(vinyl imidazole), chlorosulphonated polyolefins, latexes or polyethylene oxide.

Excipients can be added to the particle-forming solutions. Generally, excipients refer to compounds or materials that are added to ensure or increase the stability of the active agent during the spray-freeze dry or spray-freeze solvent extraction process or afterwards, for long term stability or flowability, for obtaining specific performance characteristics of a powder formulation, or other desirable characteristics of the powder product. Suitable excipients include relatively free flowing particulate solids or dissolved components that are basically innocuous when introduced into a patient and do not significantly interact with the active agent in a manner that alters its biological activity. Suitable excipients include proteins such as human or bovine serum albumin, gelatin, immunoglobulins, etc.; carbohydrates including monosaccharides such as galactose, D-mannose, sorbose, etc.; disaccharides such as lactose, trehalose, sucrose, etc.; cyclodextrins or polysaccharides such as raffinose, maltodextrins, dextrans, etc.; amino acids such as monosodium glutamate, glycine, alanine, arginine or histidine; as well as hydrophobic amino acids such as tryptophan, tyrosine, leucine, phenylalanine, etc.; a methylamine such as betaine; an excipient salt such as magnesium sulfate; a polyol such as trihydric or higher sugar alcohols, e.g. glycerin, erythritol, glycerol, arabitol, xylitol, sorbitol, or mannitol; propylene glycol; polyethylene glycol; Pluronics; surfactants; inorganic salts such as NaCl, KCl or CaCl₂; organic salts such as zinc acetate, sodium gluconate or combinations thereof. Combinations of these excipients are also possible. Suitable plasticizers include glycerin, polyethylene glycol, propylene glycol, triethyl citrate, diethyl phthalate, dibutyl phthalate, dibutyl sebacate, sorbitol, triacetin, or their combinations.

The type and concentration of polymer can be important and can be optimized in order to improve the encapsulation efficiency and particle formation. A combination of two or more different polymers can be used to optimize particle formation. Alternatively, different molecular weights of the same type of polymer can be used in order to optimize particle formation or performance. The apparatus has the ability to process higher molecular weight polymers or less-soluble components by diluting them in a greater quantity of solvent for forming the solution and enabling atomization through a wide variety of nozzles, and subsequently utilizing the warm-gas region prior to freezing to accomplish some level of evaporation to obtain the desired final particles morphology.

Any solvent, solvent mixture, or solvent/antisolvent mixture that can dissolve, disperse or form an emulsion with the chosen particle-forming mixture such that the mixture forms a solid particle when passed through spray atomization equipment into a collection fluid can find use in the present system. The liquid solvent used for the preparation of the suspension/solution/emulsion can encompass water or organic solvents with freezing points well above the freezing point of the temperature of the collection fluid. Solvents can be used alone or mixed so long as they are suitable for making a suspension/solution/emulsion of the particle-forming material. Suitable solvents include ethanol, methanol, tetrahydrofuran, acetonitrile, acetone, tert-butyl alcohol, N-methylpyrrolidone, dimethyl sulfoxide, N,N-dimethyl formamide, N,N-dimethyl acetanide, ethyl lactate, diethyl ether, methylene chloride, dichloroethane, ethyl acetate, isopropyl acetate, butyl acetate, propyl acetate, toluene, hexanes, heptane, and pentane, for example.

A primary piece of equipment used for accomplishing the spray-freeze process is a spray freeze chamber, an embodiment of which is illustrated in FIG. 1 as the enclosed spray chamber 10. The spray chamber can be operated in a low humidity environment. To accomplish this it can be positioned in an enclosed environment or in a room having low humidity. The humidity can be maintained at a low level throughout the procedure, which helps to minimize frost.

With reference to FIG. 1, to operate the spray chamber 10, a collection reservoir 30 can be assembled or must be present on the bottom of the spray chamber 10 and the collection reservoir filled to the desired level, L1, with a suitable particle collection fluid. In an exemplary method this is accomplished by activating the liquid nitrogen (LN2) auto-filling system to begin filling the container with liquid nitrogen. The flow of relatively warm air into chamber 10 through inlet port 40 can also be initiated. In systems having a vacuum attached to outlet port 50, a vacuum can be established and the system allowed to equilibrate for a suitable period of time to establish suitable multilayered temperature zones. During this time, the feed solution can be prepared to be fed to the atomization nozzle inlet.

Suitable particle collection fluids remain a liquid at temperatures that cause the droplets or particles to freeze and remain frozen and will not contaminate or degrade the particles. The particle collection fluid can be a liquefied gas, e.g. liquid nitrogen (boiling point −196° C.), liquid argon (boiling point −186° C.), liquid oxygen (boiling point −183° C.); a cooled solvent that can be at a temperature well below the freezing point of the solvent in the particle-forming mixture such as ethane, pentane, isopentane, propane, ethanol, isopropanol, n-propanol, or halocarbons; an antisolvent or mixtures of the above. The cryogenic collection fluid can be held statically in a vessel or mixed by any suitable means, or in an alternative embodiment, can be circulated or flowed through an appropriate vessel that is equipped with a filter to retain the particles that are formed.

In an exemplary method, once the collection reservoir 30 has been filled and cooled to the proper level, and the violent boil-off of liquefied gas has subsided, the particle-forming solution can be atomized. The particle-forming mixture can be passed into the atomizer using standard techniques. A pump or pressurized feed system can be run at any suitable rate, typically these are operated at rates that range from about 0.25 to about 100 ml/min. Operation outside this range depends on feed solution properties and scale of the system. Any suitable pumping or pressurized feed system can be used to supply feed solution to the nozzle of the present apparatus.

The particle-forming solution can then enter an atomization nozzle located at the top of the spray unit. The liquid flows through a small channel in the nozzle to the atomization nozzle tip, where it is atomized into droplets that project from the nozzle in a downward direction. Droplet formation occurs inside the contained spray chamber 10, at plane L3, as illustrated in FIG. 1. Relatively warm gas can be introduced through inlet port 40 so as to create a warm gaseous environment in the area of spray nozzle 20. Simultaneously, in a method, liquid nitrogen contained in the lower batch container area is boiling at a temperature of about −196° C., generating an extremely cold vapor flow upwards, counter-directional to the direction of the droplets, beginning generally at the plane noted L1. To limit penetration of the cold vapor layer up through the entire chamber, ports can be located at plane L2 that can be connected to a vacuum source to provide a lower pressure than inside the chamber. The vacuum effectively transports, directs or skims the cold nitrogen vapor flow out of the chamber at L2. As a result, there is a very sharp change in temperature gradient (dT/dZ, where T is temperature and Z is axial distance) across L2. A cold vapor layer between L1 and L2 is created that has a much colder average temperature and dT/dZ than the gaseous layer between L2 and L3. The distance-temperature plot in FIG. 2 is steep or discontinuous, showing an inflection point in the chamber gradient at the L2 interface (d²T/dZ²=0), which is indicative of a boundary condition between two separate energy equations of change, above and below L2. Once again, creation of these zones on either side of L2, whereby the energy equation changes, occurs within a single chamber due to the location of inlet and outlet ports and with the optional assistance of a vacuum.

Other flow streams can also assist with the operation of this system. To balance pressure within the sealed chamber, make-up gas (air or nitrogen) can be introduced at any suitable location. Suitable locations will not disrupt the gas layer that protects the spray nozzle 20 from fouling. For instance, a gas inlet can be placed at the top of the chamber through inlet port 40. The gas can be introduced through a filter to maintain sterility in an aseptic process. The gas may be heated or cooled, as desired, prior to entering the chamber or by application of a heating or cooling source to the region of the chamber above L2. The flow rate of make-up gas is either passively or actively controlled to provide a neutral or specific internal chamber pressure, and may also be used to limit the boil-up rate of the liquefied gases when they are used as collection fluids. This gas may also provide thermal regulation within the region of the chamber above L2 to improve the operating conditions of the nozzle. Together, the mass flow of liquid gas due to boil-up (or flow of cold inlet gas in alternate embodiments) and the mass flow of make up gas can equal the mass flow withdrawn from the chamber by application of the vacuum. The vacuum flow stream can be regulated so as to not induce high-velocity flows or turbulence at the interface that may impede or misdirect the droplets as they fall into the cold vapor or the collection fluid.

Finally, when collection fluid drops below a desired level replacement collection fluid can be introduced into the collection reservoir to replenish the level in the batch container region so the collection fluid level is maintained near L1. This serves to maintain a steady-state temperature gradient and thickness of the cold vapor layer throughout the process. Any common method of level detection and control may be used to control the level of collection fluid at its intended level.

Thus, in the embodiment of FIG. 1, the enclosed spray chamber can be used to provide three distinct temperature zones. The first, above L2, is a relatively warmer gas temperature zone formed by the passage of a gas between inlet 40 and outlet 50. This gas zone is conducive to atomization of the liquid feed and operation of the nozzle. Specifically, between L2 and L3, droplets are formed and fall through a temperature gradient that may be controlled so as to warm or cool, but not freeze or cause significant precipitation in the droplets; volatile solvents may also evaporate from the droplet surface in this region, providing another level of control to particle or process engineering. For consistent nozzle operation, the temperature at and above L3 can be kept above the freezing point of the feed solution and within the temperature range tolerated by the nozzle itself.

The second temperature zone between L2 and L3 can be a colder gaseous temperature zone formed by a cold gas that can result from a boil off of liquefied gases, or by a gas flow between outlet 50 and an inlet between outlet 50 and the surface of the collection fluid. Alternatively a cold gas could be introduced by sparging through a non-boiling cryogenic collection fluid of entirely different chemical composition (FIG. 3). Depending on: (1) the thickness of the cold vapor layer, the temperature gradient and thermal properties of the gas in this zone, and (2) the velocity, mass flow, and thermal properties of the droplets, the droplets may be frozen, congealed or cooled while traveling through this zone.

Finally, the third temperature zone is formed by the cold liquid volume below plane L1. This zone is primarily intended to ensure that droplets are frozen and remain in a solid or frozen state throughout the duration of the atomization process until the frozen droplets are extracted with an anti-solvent in a downstream process. Liquid nitrogen is a non-solvent for components in most formulation feed solutions, thus it will only freeze these components. Alternate embodiments utilize extremely cold anti-solvents such as pentane, isopentane, or ethanol that may not only freeze the droplets, but could also potentially act as an extraction solvent for the solvent in the feed solution later in the process.

The disclosed apparatus can be used to provide a warmer gas zone which facilitates consistent performance of the nozzle and enables atomization of feed solutions containing lower solids contents, which can be caused by solubility limitations, feedstock concentration limitations from an upstream process, feed viscosity limitations set by the nozzle, etc. In addition, in this zone, a volatile solvent in the droplets can partially evaporate prior to reaching the colder gas zone. The resulting loss of solvent by evaporation can be used to pre-chill the droplet as it travels away from the nozzle. In addition, a portion of this evaporated solvent can be transported out of the system through the vacuum ports at L2, thus reducing solvent load in the downstream extraction process.

The cold gaseous zone may be engineered to partially or entirely freeze the droplets. This can be particularly useful with feed solutions of low freezing-point solvents when it is desired to atomize directly into a cold extraction solvent. In this scenario, a cold vapor zone can be created that pre-chills or freezes the droplets prior to contacting the collection fluid. This reduces the rapid burst of extraction on contact with the extraction of feed solvent that can often result in undesirable particle morphology. Moreover, a cold zone can help to avoid the use of LN2 as a collection fluid. LN2 is notoriously difficult to use in aseptic processes because of the fact that it is generated in industrial settings. Consequently, products made with LN2 can be difficult to validate. Embodiments of the apparatus can utilize a sterile-filtered, chilled gas in the cold gas zone to effectively eliminate LN2 from the process.

In an embodiment a plurality of spray chambers can be assembled in parallel. Such a device can be used to simultaneously create a series of particle formulations in a high throughput manner such that formulation development time can be reduced. The spray chambers can be any disclosed spray chamber. Thus, spray chambers can include a spray nozzle and a collection reservoir. They can also include a gas inlet and a gas outlet and can have a spray nozzle located in a gas layer at about or above the level of the gas outlet. The collection reservoir can be located below the level of the gas outlet. In an embodiment the spray chambers can be joined to a common utility system using, for example, distribution manifolds and valves. In an embodiment, batch production can be automated. To this end one or more batch production systems can be connected to a central control system that can be used to generate a series of formulations that can be prepared, processed in the chambers, and tested.

FIG. 20 illustrates an embodiment of a system containing multiple spray chambers 110. As illustrated, the system can be configured with a common utility system such that a central cooling system, such as a chiller and/or vaporizer 120, can provide cooling to one or more jackets 130 that surround each chamber 110. A central vacuum drying system 140 can also be configured to supply a vacuum to one or more of the spray chambers 110. The system can also be configured with waste vessels for collecting various waste materials that are generated in carrying out the disclosed methods. Further, the system can be configured with a gas sparge feed line that can be used to sparge gas into one or more of the chambers 110, as desired. Some or all of the valves, sensors, and other devices within the system can be integrated into a centralized control system, and control logic applied to enable process monitoring and automation of each spray chamber, independent of the others, throughout the particle production process. It can be appreciated that many variations of this system are possible. For example, one or more vacuum drying systems 140, chillers or vaporizers 120, and gas sparge feed lines 160 could be dedicated to individual chambers 110. Additional liquid or gas streams within the system can be joined to additional common utility systems. The system comprising a plurality of spray chambers can be integrated into an enclosed environment, with the enclosure system maintaining, for example, a low humidity environment, and providing structural support for the spray chambers, associated piping and utility systems.

In one method, once the particle-forming solution has been completely atomized, the liquid pump and atomizer power are shut off. When present, the auto-fill system for the collection reservoir is de-activated. The collection reservoir (batch container) can then be de-coupled from the bottom of the spray chamber, the collection fluid removed and particles concentrated. The collection fluid can be removed by any suitable method. For example, in methods that use liquefied gases, the collection fluid can be allowed to simply boil off or can be filtered away. Alternative embodiments of the system could leave the container in place and remove the bulk of the LN2 by evaporation and/or selective filtration when filters are present. Anti-solvent based collection fluids can be quickly removed by selective filtration.

The particles can then be extracted with an anti-solvent to remove the polymer solvent from the solid frozen particles. Any cryogenic extraction method that extracts solvent from the particles without damaging the particles can be used. Solvent extraction can be conveniently carried out under temperature-controlled conditions, in a cryogenic temperature bath, or by circulating a cooling-fluid through a jacketed collection vessel or through a heat exchange surface within the vessel, for example. Care must be used when the method involves exchange of an extraction solvent for a liquefied gas. It is important to accurately time the cold extraction solvent addition to near the end of the liquefied gas boil off, as addition of the solvent to the batch container can lead to violent boil-off of the liquefied gas and loss of the nascent particles. If the liquefied gas completely boils-off, before extraction solvent (anti-solvent) is added, the particles may thaw and agglomerate.

Any suitable anti-solvent can be used to extract the particle-forming material solvent from the nascent particles. As used herein, the term “anti-solvent” refers to a solvent or solvent mixture that does not substantially dissolve the particle-forming material in the nascent particles but is miscible with the solvent in the microparticles. It can be appreciated that a suitable anti-solvent will depend on the nature of the particle-forming material and its solvent and it is well within the skill of one having skill in the art to select such an anti-solvent. The anti-solvent can be added in excess so as to ensure efficient and complete extraction of solvent from the particles. The particles can be extracted and then collected by filtration, washed, and re-extracted until solvent removal is complete. One suitable anti-solvent is pentane which can be chilled to about −120° C. As pentane is first introduced to a batch container and exchanged with liquefied gas, the remaining liquefied gas vaporizes quickly. The temperature of the nascent particles can be maintained below the freezing point of the polymer solvent (−96.7° C. for CH₃Cl, −83° C. for Ethyl Acetate) during the initial stage of the extraction process, which may be as little as 5 minutes or shorter. A thermocouple meter or other temperature gauge can be used during the extraction to ensure that suitable temperatures are maintained during the extraction.

Shortly after anti-solvent addition, the temperature can be allowed to increase from the initial temperature of the extraction anti-solvent. However, no warming needs to take place and warming need not be to a temperature above the melting point of the solvent in the particle for the extraction to be effective. A mixer can be used in each particle collection reservoir to maintain a homogeneous temperature and chemical composition during extraction. The polymer solvent is extracted into the anti-solvent, resulting in the formation of more stable solid particles containing the polymer, active compound and any additional excipients. Microparticles can be kept in a temperature bath and extracted for extended periods, from 4 to 72 hours, prior to filtration and drying. However, extraction times of much shorter duration are also feasible, for as little as 30 minutes to 2 hours, depending on the equipment design, temperatures, and solvent systems.

Microparticle filtration occurs after a sufficient period of time to form stable solid particles. Filtration and rinsing of the particles can be accomplished using temperature-controlled cold anti-solvents and chilled equipment, to prevent the solvents from having negative effects on the particles should a decrease in the polymer glass transition temperature or other temperature-dependent stability issue be attributed to the presence of the solvents. In one method, once the filter cakes are obtained, they can be transported to a shelf lyophilizer for residual solvent removal under vacuum using standard techniques. In general, the vacuum cycle begins at a low temperature (for example, at −40° C.) which increases over several hours to ambient temperature. Alternatively, particles may be dried within the same device used for filtration, as in, for example, a Sweco PharmaSep™ unit, or, in any other common batch or continuous particle drying device. At the end of the cycle, the particles are removed and collected and stored until they are analyzed, packaged or otherwise used.

The spray chambers of the invention can be conveniently used in conjunction with a variety of additional process equipment. For example, the spray chamber and associated equipment can be placed in a bench-top or free-standing enclosure system, which can have aluminum structural framing, stainless steel panels, and plexi-glass windows, for example. The enclosure system can provide structural support for utilities such as pressurized air, vacuum, liquefied nitrogen and associated piping and can be used to maintain a low humidity environment inside to minimize frost on cold surfaces. Alternatively, the spray chamber and associated equipment can be located in a potent containment or aseptic isolator.

A liquid nitrogen supply system comprising LN2 cylinders can be used to supply a cryogenic temperature bath, anti-solvent chiller and the spray-freeze unit with liquid nitrogen. Vacuum-jacketed or insulated LN2 tank supply hoses can be connected to a distribution manifold fitted with solenoid valves to control distribution of LN2 to each piece of equipment.

A cryogenic temperature bath comprising a vacuum-jacketed or insulated temperature bath can be used to keep collection reservoirs cold during the extraction phase of the process. The minimum operating temperature of the bath is determined by the temperature limitations of the cooling system, for example LN2 or chilled water cooling systems, and the heat transfer fluids used. The bath can have any number of wells for storing particle collection reservoirs, which can be removed from the spray-freeze chamber after atomization and stored in the temperature bath until processing. A control system can regulate LN2 or other coolant flow for maintaining the bath temperature and can be used to control power to batch mixers inserted into the collection reservoirs during extraction.

In one method, a cryogenic solvent chiller comprising a vacuum-jacketed or insulated solvent chiller and having a 6-liter capacity, enabling a ready cold solvent supply, can be used to cool extraction solvent to its minimum temperature (approximately −120 to −125° C. for pentane; approximately −150° C. for isopentane) prior to its addition to the collection reservoir containing frozen micro-droplets. A control system can also be used to regulate LN2 flow or other coolant for maintaining solvent temperature and controlling power to the chiller mixer.

In an embodiment, collection reservoirs can be 4 inches in diameter and have a domed bottom, and an 800-ml capacity. Multiple batches may be produced in a single run with each batch in a separate particle collection reservoir. Reservoirs can be placed into slots in the cryogenic temperature bath following atomization and stored for downstream processing. Batch mixing heads can contain an impeller-type mixer system, a thermocouple for monitoring batch temperature, and ports for solvent addition and venting can also be used to assist in the extraction procedure.

Filtration equipment can be used to recover particles. This equipment can include a filter housing configured to hold a filter and can be removed from the filtration setup following filtration and covered with a venting cap. A filter capsule can be designed to be conveniently placed into a vacuum drying system or into a shelf lyophilizer to remove residual solvents under controlled temperatures.

In a method the disclosed spray freeze solid extraction equipment can be used to prepare amorphous solid dispersions of poorly aqueous soluble molecules.

### EXAMPLE 1

The following example describes an exemplary procedure for making microparticles with the embodiment illustrated in FIG. 1.

Initially, the humidity in the environment of the microparticle formation equipment is reduced to about 15% relative humidity and maintained at this level to the extent possible to minimize frost.

A cryogenic temperature bath is then started up to pre-cool it to the intended steady-state particle extraction temperature. The cooling-source for the bath is liquid nitrogen, which is applied through a copper-coil fixed within the bath. Heat transfer fluid within the bath provides contact between the cooling coil and wells that hold particle collection reservoirs. A mixer within the bath fluid circulates the fluid to maintain a homogeneous temperature. After the mixer is started, a temperature bath control system is activated and the set-point confirmed to begin flow of liquid nitrogen to the bath to begin cooling it. As the initial temperature is above the set point, the controller will signal a solenoid to open to begin this flow. Cooling of the temperature bath to −83° C. takes approximately 4 hours from initialization. While waiting for the bath to reach operating temperature, ethanol is added to each well in the bath to provide thermal contact between the well and the particle collection reservoirs that will be placed in them later in the process.

The cryogenic solvent chiller is then started for pre-cooling the extraction solvent to just above its freezing temperature. The cooling-source for the vessel is liquid nitrogen, which is applied through a stainless steel coil fixed within the vessel. Pentane (MP −129° C.) is transferred from a pressure vessel to the chiller vessel by connecting a transfer line from the pressure vessel to the chiller. Vapors generated in the solvent chiller during this transfer are vented by separate connection of a vacuum line to a port at the top of the chiller vessel. After filling, a mixer in the solvent chiller vessel is started. The solvent chiller control system is activated and the temperature set point confirmed to begin flow of liquid nitrogen to the vessel to begin cooling the pentane. As the initial pentane temperature is above the set point, the controller signals a solenoid to open to begin the flow of LN2 into the solvent chiller. Cooling of the solvent chiller filled with 6-liters of pentane, to −122° C. takes approximately 30 minutes from initialization.

Next, the spray freeze chamber is prepared for operation. Liquid nitrogen (LN2) used in the spray freeze chamber during the spray freeze portion of the process is taken from the same supply tank as that used for the solvent chiller. A separate solenoid valve controls flow from the LN2 tank to the flow line leading to the spray freezing chamber. The controller regulating LN2 flow to the spray freeze chamber is activated. Upon activation, the temperature controller will indicate the thermocouple probe temperature of the batch container (collection reservoir) at L1, FIG. 1. A set point of −184° C. is input into the controller when LN2 is the collection fluid. As the initial temperature is above the set point, the controller signals the solenoid to open and the particle collection reservoir is filled. Subsequently, during the spraying operation, LN2 is delivered automatically by the controller throughout the course of the run.

As described previously, vacuum ports on the spray freeze chamber can be used to remove nitrogen gas boil-off during the spraying process, to prevent excessive cooling of the nozzle. Vacuum can be supplied to the enclosure through the back panel, and can be connected to the spray freeze chamber. A needle valve on the spray freeze unit can be used to control vacuum to the spray chamber. Initially, the valve can be set such that the vacuum gauge on the downstream side of the valve reads −21 in. Hg.

A chamber light on the spray freeze unit enables the user to view the spray pattern and LN2 level within the chamber through a window on the spray chamber. This can be activated prior to beginning processing of the batch.

Prior to initiating atomization, a batch container (collection reservoir) can be assembled onto the bottom of the spray chamber. The liquid nitrogen (LN2) auto-filling system can be activated to begin filling the container with liquid nitrogen. During this time, the feed solution can be loaded into a pre-rinsed syringe and the syringe attached to a syringe pump. A feed tube can be attached from the syringe hub and the atomization nozzle inlet. Parameters can be set to regulate the pump rate (ml/min) and duration; the pump can be set to operate at 0.5-1.0 ml/min.

Once the particle collection reservoir in the spray chamber has been cooled and filled to the proper level and the violent boil-off of LN2 has subsided, the feed solution is ready to be atomized. Power to an ultrasonic nozzle, such as those produced by Sono-Tek Corp. (Milton, N.Y.) can be enabled and the power reading on the nozzle control box can be set. The syringe pump can be immediately activated to begin pumping solution to the nozzle tip. Initially and periodically during the spray process, an operator can inspect the spray for consistency in spray pattern and can tune the power sent to the nozzle as necessary to maintain a suitable spray pattern. During spraying, the cold gas (vapor) zone becomes visible at the interface L2 (FIG. 1) as the evaporated polymer solvent from the droplets re-condenses on-contact with the cold gas. Flow of this gaseous layer is visible as it flows in a laminar manner out through the vacuum ports. The droplets themselves generate minor perturbations in the center of this layer, as the droplets fall through it and into the liquefied gas collection fluid, where they remain frozen until extraction solvent is added. Periodically throughout the course of the run, the LN2 auto-Fill system will actuate to top-off the LN2 level in the particle collection reservoir. The LN2 re-filling operation can be done in place, without interrupting the spraying operation or lowering the particle collection reservoir.

Once the microparticle-forming feed solution has been completely atomized, the syringe pump and atomizer power can be shut off. The LN2 auto-fill system can be de-activated and the particle collection reservoir de-coupled from the bottom of the spray chamber. The particle collection reservoir can be partially covered and set aside to allow LN2 to boil off.

To prepare for addition of the extraction solvent, particle collection reservoirs can be placed into the cryogenic temperature bath after about ½ of the LN2 has evaporated from the reservoir. Boil-off of LN2 can be carried out over approximately 20 to 40 minutes once the batch has been placed into the bath. The liquid level can be monitored carefully to accurately time cold extraction anti-solvent addition. If the cure solvent is added prematurely, violent boil-off of LN2 may occur, making solvent addition difficult. Freezing of the extraction solvent may also occur. If the LN2 completely boils off prior to extraction solvent addition, the particles may thaw and agglomerate.

As the LN2 level reaches the vicinity of the particles, the batch mixer can be assembled. A thermocouple probe on the mixer head can be attached to the thermocouple meter junction box.

When the LN2 level is reduced to an acceptable level, the cold extraction solvent can be added. The batch mixer motor can be activated prior to placing the mixer head into the particle collection reservoir. It can be helpful to activate the agitator before adding the extraction solvent to ensure mixing during solvent addition. The fill tube from the solvent chiller can be primed into a waste container to pre-chill it, and then can be inserted through a port on the mixer head.

Immediately after inserting the tube, the solvent chiller bottom valve can be opened to begin adding pentane to the particle batch. The pentane, chilled to −120° C., will warm slightly as it is added. As pentane is first introduced to the batch container, the remaining LN2 vaporizes quickly. Timed addition of the pentane cure solvent has been used to fill the batch container and a level-stick has been used to top off the extraction solvent level (typically 600-700 ml) after the initial timed-fill. After the solvent addition is complete, the solvent chiller valve can be closed and the fill tube removed from the particle collection reservoir. The temperature of the particles can be maintained below the freezing point of the polymer solvent (−96.7° C. for MeCl, −83° C. for ethyl acetate) during the initial stage of the extraction process, which may be as little as 5 min. The thermocouple meter can be observed or electronically recorded during and just after solvent addition and maximum and minimum initial temperatures can be noted in a batch sheet when appropriate.

Following solvent addition, the batch temperature begins to increase from the initial temperature of the extraction solvent from the solvent chiller, typically −120° C., to the set-point temperature of the cryogenic temperature bath which can be in the range of about −80° C. to 5° C., generally. The mixer in each particle collection reservoir keeps the temperature and chemical composition of the extraction solvent spatially uniform during extraction. The polymer solvent can be extracted into the curing anti-solvent, resulting in the formation of stable solid product particles containing the particle-forming material mixture. Microparticles are typically kept in the cryogenic temperature bath for 4-18 hours prior to filtration and drying. However, extraction times of longer duration (18 hours to 72 hours) or much shorter duration can also be used. As little as 30 minutes to 2 hours can be used depending on the equipment design, temperatures, and solvent systems.

Microparticle filtration occurs after particle batches have been extracted for a sufficient period of time to form stable solid particles. This is typically carried out the day following spraying and extraction initiation. Filtration and rinsing of the particles can be accomplished using cold pentane and chilled metallic equipment, to prevent the solvents from having negative effects on the particles should a decrease in the polymer glass transition temperature or other temperature-dependent stability issue be attributed to the presence of the solvents

The filtration assembly can consist of a filtration manifold, to which a gasket can be placed onto one of the manifold's concentric reducers. A stainless steel filter capsule can be placed on top of the gasket and secured with a clamp. The filter capsule contains a stainless steel filter element that allows passage of the solvent but retains the microparticle product. The filter and filter capsule also can be placed into the lyophilizer where the permeable filter permits removal of residual solvents. A second gasket can be placed on the top flange of the filter capsule, followed by a jacketed stainless steel funnel to complete assembly of the filtration set-up. Dry ice may be placed in the area between the funnel and the outermost wall of the stainless steel container surrounding it to pre-chill the funnel prior to filtration.

To initiate filtration, cold solvent (−120° C. pentane) can be transferred from the solvent chiller into a bottle. Some of this solvent can be added to the filtration funnel to pre-chill and wet it. The mixer for the batch to be filtered can be then shut down and the batch container removed from the cryogenic temperature bath. At the filtration station, approximately ½ of the curing anti-solvent-microsphere solution from the particle collection reservoir can be poured into the filtration funnel. The remaining batch contents are then gently agitated in the particle collection reservoir to maintain the particles in a suspended state prior to adding the solution to the funnel. A vacuum can be activated on the manifold to pull the solvent through the filter and into a collection vessel, leaving a filter cake in the filtration capsule. The cake can be rinsed several times with cold, clean pentane. Immediately after the last rinse has been performed and the cake runs dry, the vacuum can be deactivated to prevent excessive air from being drawn through the cake, which could warm it.

After filtration and rinsing, the filtration assembly can be disassembled. A vent lid can be placed onto the filter capsule and clamped in place. The capsule can then be transported on dry-ice to the lyophilizer and placed on a pre-chilled shelf. Once all capsules have been loaded into the lyophilizer, the lyophilizer can be sealed and a vacuum cycle can be activated to remove the residual solvents from the wet cake. Generally, the vacuum cycle begins at a low temperature (−40° C.) and ramps over several hours to ambient temperature. At the end of the cycle, capsules are removed and the particles collected and stored until analyzed or used.

### EXAMPLE 2

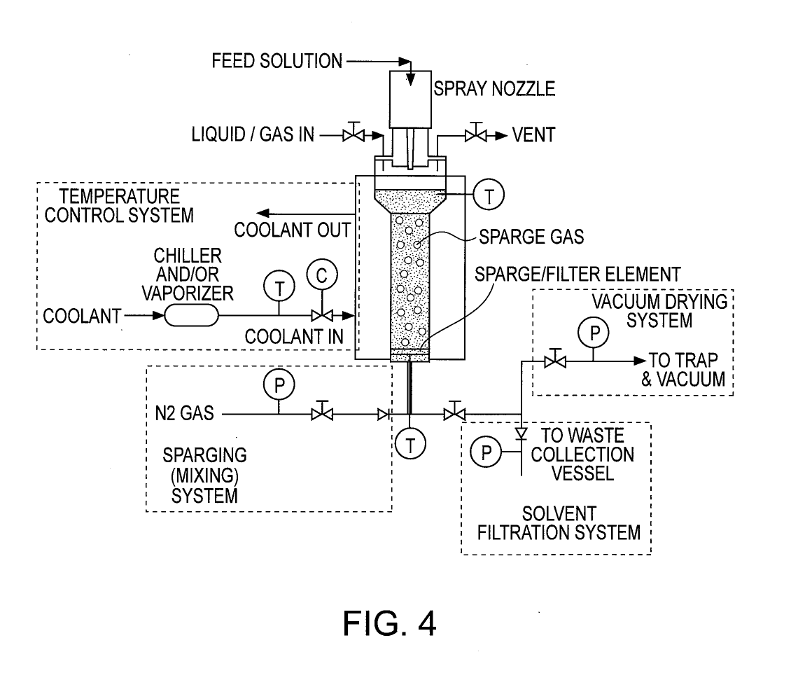

The following example provides a procedure for making microparticles with the embodiment illustrated in FIG. 3, FIG. 4 or FIG. 20. In this example, atomization is performed directly above a cold extraction solvent; sparging through the cold solvent provides an up-flow of chilled nitrogen gas that can pre-chill or freeze the droplets prior to contacting the cold solvent. In this example, liquefied gas, such as liquefied nitrogen, is not utilized as a freezing non-solvent.

Initially, the humidity in the environment of the microparticle formation equipment is reduced to about 15% relative humidity and maintained at this level to the extent possible to minimize frost.

One or more cryogenic chillers are then started up in preparation for supplying coolant to the outside jacket of the collection reservoir. A liquefied gas vaporizer and refrigerated gas chiller have been used in the present example, but other types of cryogenic chillers, such as a recirculating liquid chiller, are also possible. The liquefied gas vaporizer in the present example is pre-chilled by beginning the flow of liquid nitrogen to the vaporizer, and initiating the vaporizer temperature controller to produce cold nitrogen gas through application of heat. The refrigerated gas chiller in the present example is pre-chilled by initiating start-up of the chiller's closed refrigeration cycle and waiting for the system to equilibrate before beginning flow of ambient-temperature nitrogen or dry air. Thus, in either case of the present example, cold gas is intended as the coolant to be applied to the exterior of the collection reservoir.

Once the cryogenic chillers have been pre-chilled, ambient extraction solvent, such as n-pentane (MP −129° C.), is added to the collection reservoir in FIG. 3. Temperature monitoring within the solvent (“T” in FIG. 3 and FIG. 4) in the collection reservoir is established, the temperature control system is activated and the temperature set point confirmed. Temperature may be monitored at multiple points within the collection reservoir; one, multiple or an average of these values may be used for control. Initiating temperature control begins the flow of coolant to the jacket surrounding the collection vessel containing extraction solvent. In this particular example, the solvent is first cooled to an intermediate temperature of approximately −40° C. using a gas chiller, where it is held while preparing for atomization. At a point during the cooling of the solvent, ambient nitrogen gas is supplied to the sparge-gas inlet at the bottom of the collection reservoir. As the sparge gas exits the sparging element, small gas bubbles are produced, which cool as they rise through the extraction solvent. The sparge gas, in this manner, not only acts to homogenize the temperature and concentration gradients within the collection reservoir by means of the kinetic movement of the bubbles, but, upon reaching the surface, produces a net up-flow of chilled nitrogen gas useful for pre-chilling or freezing the atomized droplets.

As described previously, venting ports on the spray freeze chamber are used to remove the nitrogen sparge gas up-flow during the spraying process, to prevent excessive cooling of the nozzle. These venting ports may alternatively be connected to a regulated vacuum source, as previously described. In the present example, the nozzle tip is located at or above the position of the venting ports

A chamber light on the spray freeze unit enables the user to view the spray pattern within the chamber through a window on the spray chamber. This is also activated prior to beginning processing of the batch.

Prior to initiating atomization, the coolant source is switched to the liquefied gas vaporizer, which produces gaseous nitrogen at temperatures sufficiently cold (approximately −170° C.) to rapidly reduce the extraction solvent temperature. In the present example, the temperature set-point for the pentane extraction solvent is adjusted to approximately −122° C., just above the melting point of n-pentane, and a control valve regulates flow of the cold nitrogen coolant to the exterior of the collection reservoir to maintain the temperature. During this time, the feed solution is loaded into a pre-rinsed syringe and the syringe attached to a syringe pump. A feed tube is attached from the syringe hub and the atomization nozzle inlet. Parameters are set to regulate the pump rate (ml/min) and duration; in the present example, the pump is set to operate at 0.5-1.0 ml/min.

Once the particle collection reservoir in the spray chamber has been filled to the proper level with extraction solvent, sparging established, and the solvent chilled to its temperature for freezing or maintaining frozen droplets, the feed solution is ready to be atomized. Power to a Sonotek™ ultrasonic nozzle is enabled and the power reading on the nozzle control box is set. The syringe pump is immediately activated to begin pumping solution to the nozzle tip. Initially and periodically during the spray process, an operator can inspect the spray for consistency in spray pattern and can tune the power sent to the nozzle as necessary to maintain a suitable spray pattern. Temperature monitoring and control of the chilled extraction solvent is maintained throughout the atomization process by application of coolant to the collection reservoir; in the case of n-pentane, this temperature is maintained at approximately −122° C.

Once the microparticle-forming feed solution has been completely atomized, the syringe pump and atomizer power are shut off. The temperature control of the extraction solvent within the collection reservoir may then be maintained at or adjusted above the initial temperature for atomization for accomplishing extraction of the solvent. Pre-programmed, multi-step ramp-and-hold functions are possible through the use of an integrated control system. Typically, temperature is maintained only a short period of time at the temperature for freezing (approximately −122° C. for n-pentane) after atomization has terminated, before allowing for a ramp over approximately 15-120 minutes to a steady-state temperature in the range of −80° C. to ambient, generally. Coolant, supplied to the exterior of the collection reservoir, can be used during the ramp; alternatively the temperature can be allowed to rise passively.

In this example, the nitrogen sparging within the particle collection reservoir keeps the temperature and chemical composition of the extraction solvent homogeneous during extraction. The polymer solvent is extracted into the curing anti-solvent, resulting in the formation of stable solid product particles containing the particle-forming material mixture. Microparticles are typically kept in the collection reservoir, under temperature control, for 30 minutes-18 hours prior to filtration and drying. Additional extraction solvent or an extraction co-solvent may be added during the curing process to alter the miscibility properties between polymer solvent and the extraction solvent, or to make up for solvent lost to evaporation.

Microparticle filtration occurs after particle batch has been extracted for a sufficient period of time to form stable solid particles. In this example, filtration, rinsing and drying can be carried out in situ within a suitably designed collection reservoir (FIG. 4). Sparging of the extraction solvent is halted, the sparging nitrogen supply valve is closed, and a second valve off the bottom port on the collection reservoir is opened to access a solvent collection vessel. During the filtration, the sparging element acts as a filter to permit passage of solvent while retaining the microparticles. Rinsing of the filter cake follows. Filtration and rinsing of the particles is typically accomplished while maintaining temperature control of the collection reservoir, to prevent temperature from having negative effects on the particles should a decrease in the polymer glass transition temperature or other temperature-dependent stability issue be attributed to the presence of the solvents. A vacuum source may be used downstream of the filter, or pressurized gas may be used upstream of the filter, to assist with filtration and rinsing.

In this example, after filtration and rinsing, vacuum drying of the microparticle cake can also be carried out in situ with a suitably designed collection reservoir and atomization chamber. All valves to the atomization chamber and collection vessel are initially closed; valve(s) downstream of the filter/product cake, leading to a high vacuum source with solvent condensing system, are then opened (FIG. 4, FIG. 20). A vacuum cycle is activated to remove the residual solvents from the wet cake. Generally, the vacuum cycle begins at a low temperature (−60 to 5° C.) and ramps over several hours to higher temperatures. Temperature control of the collection reservoir and product cake is accomplished using the coolant source. At the end of the cycle, the entire vessel is vented and the particle cake removed, collected into a separate container, and stored until analyzed or used.

### EXAMPLE 3

This example demonstrates the production of particles containing PLGA. Microparticles were prepared using the procedure described in Example 1. PLGA (5050 DL2A, Lakeshore Biomaterials) was dissolved in dichloromethane (DCM) at a concentration of 0.11 g/ml. The solution was atomized at 0.5 ml/min into a collection reservoir containing liquid nitrogen using a 25 KHz ultrasonic nozzle (SonoTek Inc.; Milton, N.Y.) at 1.4 W. Following atomization, the collection reservoir was de-coupled from the atomization chamber. LN₂ was allowed to evaporate to near completeness and chilled pentane was added at −120° C. (˜70:1 Non-Solvent:Solvent v/v) to begin extraction, while a motorized mixer was activated to stir the vessel contents. System temperature warmed to −85° C. before transferring containers to a cryogenic bath at −83° C. (DCM) for equilibration over ˜18 hours. Following extraction in n-pentane, particles were filtered and rinsed with cold pentane (−80° C.), before the filter cake was placed in a shelf lyophilizer (Virtis) pre-chilled to −40° C. Drying occurred under vacuum over 36 hours using a ramp cycle from −40° C. to 20° C.

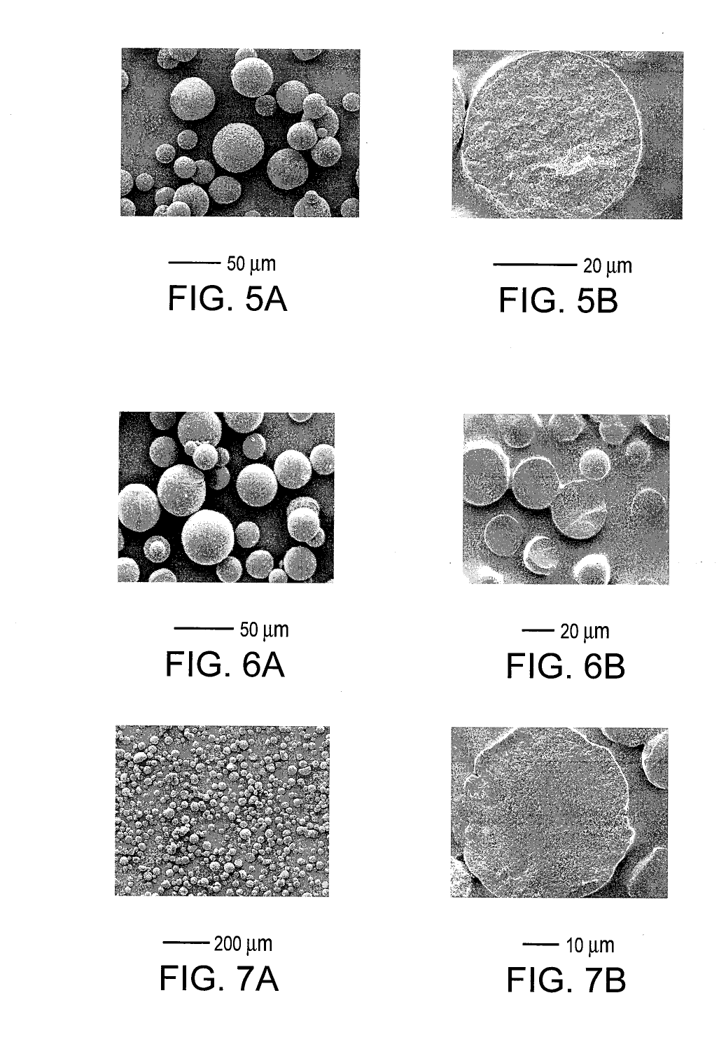

Material removed from the lyophilizer appeared as a fine, dry, white powder. SEM imaging of the powder revealed discrete microparticles with spherical shape and solid interiors (FIG. 5).

### EXAMPLE 4

This example demonstrates the preparation of microparticles that contain a peptide. Microparticles containing 10 wt % of a peptide, a bradykinin B1 receptor antagonist peptide as described in US 2005/0215470 A1 which is incorporated herein by reference, were prepared by the procedure described in Example 1. A formulation approach was utilized in which the peptide and polymer were co-dissolved in the polymer solvent. PLGA (5050 DL2A, Lakeshore Biomaterials) was dissolved in dichloroethane (DCE) at a concentration of 0.11 g/ml. In a separate vial, 179 mg of the peptide was dissolved in 0.24 ml of methanol. The polymer solution was then added to the peptide solution to form a single formulation solution.

The solution was atomized at 0.5 ml/min into a collection reservoir containing liquid nitrogen using a 25 KHz ultrasonic nozzle (SonoTek Inc.; Milton, N.Y.) at 1.4 W. Following atomization, the collection reservoir was de-coupled from the atomization chamber and placed in a cryogenic temperature bath at −30° C. LN₂ was allowed to evaporate to near completeness and chilled pentane was added at −120° C. (˜100:1 Non-Solvent:Solvent v/v) to begin extraction, while a motorized mixer was activated to stir the vessel contents. System temperature warmed to −30° C., where it was held for ˜18 hours. Following extraction in n-pentane, particles were filtered and rinsed with cold pentane (−80° C.), before the filter cake was placed in a shelf lyophilizer (Virtis) pre-chilled to 40° C. Drying occurred under vacuum over 36 hours using a ramp cycle from −40° C. to 20° C.

Material removed from the lyophilizer appeared as a fine, dry, white powder. SEM imaging of the powder revealed discrete microparticles with spherical shape and solid interiors (FIG. 6).

### EXAMPLE 5

This example demonstrates the production of microparticles containing 5 wt % of a peptide, a Bradykinin B1 receptor antagonist peptide as described in U.S. Pat. No. 5,834,431 and US 2005/0215470 A1, using the procedure described in Example 2 and the apparatus of FIG. 3, FIG. 4 or FIG. 20. A formulation approach was utilized in which the peptide and polymer were co-dissolved in the polymer solvent. A 0.10 g/ml solution of a 50:50 PLGA polymer (RG502H polymer, Mₙ˜4232, IV˜0.16 dL/g; Boehringer Ingelheim Corp.) in dichloroethane (DCE) was prepared by weighing 0.56 g of polymer into a 40-ml vial. 5.6 ml of dichloroethane was added to dissolve the polymer. In a separate vial, 35 mg of the peptide was weighed and 0.23 ml of methanol added. The polymer solution was then added to the peptide solution to form a single formulation solution.

140 ml of n-pentane was added to the collection reservoir at room temperature. The pentane was then chilled and maintained at a target temperature of −122° C. The vaporized liquid nitrogen used in the cooling system was supplied at −150° C. Sparging of N₂ gas was initiated at ˜50 sccm.

Approximately 2 ml of the formulation solution was loaded into a syringe and spraying was initiated at a flow rate of 0.5 ml/min and an atomization power of 1.4 W using a Sono-Tek™ 25 kHz nozzle. Following atomization, temperature of the pentane was maintained for 5 minutes at −122° C. before the set point was adjusted to −30° C. Sparging remained continuous throughout the curing stage. The particles were cured at −30° C. for 21 hours. Prior to filtration, the vacuum system cold trap was prepared; sparging to the reactor was deactivated. Pentane was removed using a positive pressure filtration at 40 psi, forming a filter cake of particles at the bottom of the collection reservoir. Fresh pentane, chilled to −80° C. was used to rinse the cake. Following the rinse, the temperature set point for initiating low-temperature vacuum drying was adjusted to −40° C. (measured from the bottom of the collection reservoir, as shown in FIG. 4), and the vacuum drying was started.

After approximately 4.5 hours, the vacuum system had achieved a pressure of less than 0.1 mT. At this time, the temperature set point was adjusted to 20° C. Vacuum drying continued for an additional 20 hours.

Material removed from each reactor appeared as a fine, dry, white powder. SEM imaging of the powder revealed discrete microparticles with semi-spherical shape and solid interiors (FIG. 7).

### EXAMPLE 6

This example demonstrates the production of microparticles using the procedure described in Example 2 and the apparatus of FIG. 3, FIG. 4 or FIG. 20. PLGA (5050 DL2A, Lakeshore Biomaterials) was dissolved in dichloroethane at a concentration of 0.11 g/ml. The solution was atomized at 0.5 ml/min directly into a temperature-controlled collection reservoir containing iso-pentane at −152° C., using a 25 KHz ultrasonic nozzle (SonoTek Inc.; Milton, N.Y.) at 1.4 W. A custom-built system, as illustrated in FIG. 4, was used for containing the spray and for precisely controlling the temperature of the extraction non-solvent throughout the freezing and extraction process. Nitrogen gas sparging at ˜50 sccm was applied within the collection reservoir during freezing and extraction to homogenize the contents. Following atomization, the non-solvent to solvent ratio was ˜70:1 v/v; temperature was ramped to −30° C. over ˜45 minutes before equilibration for 4 hours. Particles were filtered in place and rinsed with cold pentane (−80° C.) before drying under vacuum over ˜18 hours using a ramp cycle from −30° C. to 20° C.

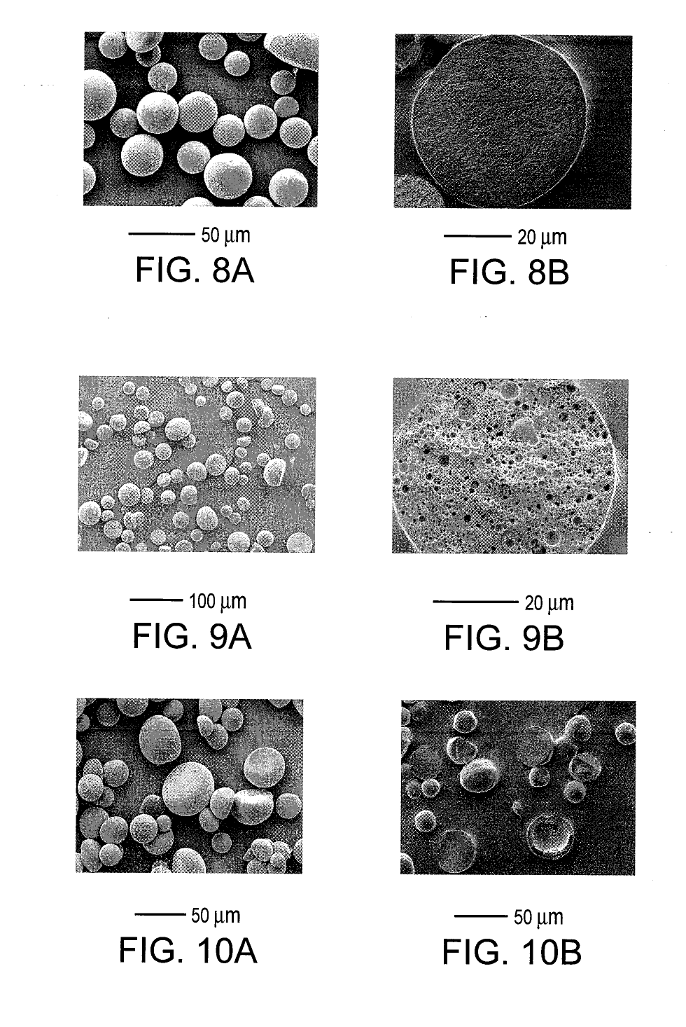

Material removed from each reactor appeared as a fine, dry, white powder. SEM imaging of the powder revealed discrete microparticles with semi-spherical shape and solid interiors (FIG. 8).

### EXAMPLE 7

This example demonstrates the preparation of microparticles containing 3 wt % of peptide (porcine PYY (3-36)) using the procedure described in Example 2 and the apparatus of FIG. 3, FIG. 4 or FIG. 20, utilizing a formulation approach in which the peptide is dissolved in an aqueous phase and subsequently emulsified in the organic polymer solution prior to forming particles.

A 0.11 g/ml solution of a 50:50 PLGA polymer (5050 DL2A, Lakeshore Biomaterials) was prepared in dichloroethane (DCE). Span 85, a surfactant, was added to the polymer solution at a concentration of 1.5 mg/ml. In a separate vial, peptide was dissolved in an aqueous solution of 50 mM Phosphate Buffer (pH 7.0) at 35 mg/ml. The peptide aqueous solution was added to the polymer solution, and homogenized using an Ultra-Turrax® (IKA Works, Inc.) for 3 minutes under ice at 25000 rpm. The emulsion was further bath sonicated for 3 minutes to form a fine emulsion. The final volume ratio of DCE to water was approximately 90:10.

The emulsion was atomized at 0.5 ml/min directly into a temperature-controlled collection reservoir containing n-pentane at −122° C., using a 25 KHz ultrasonic nozzle (SonoTek Inc.; Milton, N.Y.) at 1.6 W. A custom-built system was used for containing the spray and for precisely controlling the temperature of the extraction non-solvent throughout the freezing and extraction process (FIG. 4). Nitrogen gas sparging at ˜50 sccm was applied within the collection reservoir during freezing and extraction to homogenize the contents. Following atomization, the non-solvent to solvent ratio was ˜70:1 v/v; temperature was ramped to −30° C. over ˜45 minutes before equilibration for 21 hours. Particles were filtered in place and rinsed with cold pentane before drying under vacuum over ˜18 hours using a ramp cycle from −30° C. to 20° C.

Material removed from each reactor appeared dry, fine, white powder. SEM imaging of the powder revealed discrete microparticles with semi-spherical shape and porous interiors (FIG. 9).

### EXAMPLE 8

This example demonstrates that solid core microparticles can be produced from a high-boiling point (202° C.), high melting point (−25° C.) solvent in the disclosed apparatus. Microparticles were prepared using the procedure described in Example 2 and the apparatus of FIG. 3, FIG. 4 or FIG. 20. PLGA (5050 DL2A, Lakeshore Biomaterials) was dissolved in N-methylpyrrolidone (NMP) at a concentration of 0.11 g/ml. The solution was atomized at 0.5 ml/min directly into a temperature-controlled collection reservoir containing 75:25 v/v Pentane:Ethanol non-solvent mixture at −115° C., using a 25 KHz ultrasonic nozzle (SonoTek Inc.; Milton, N.Y.) at 2.0 W. A custom-built system, as illustrated in FIG. 4, was used for containing the spray and for precisely controlling the temperature of the extraction non-solvent throughout the freezing and extraction process. Nitrogen gas sparging at ˜50 sccm was applied within the collection reservoir during freezing and extraction to homogenize the contents. Following atomization, the non-solvent to solvent ratio was ˜70:1 v/v; temperature was ramped to −5.0° C. over ˜70 minutes and equilibration for a total extraction time of 2 hours. Particles were filtered in place and rinsed with cold pentane (−80° C.) before drying under vacuum over ˜18 hours using a ramp cycle from −20° C. to 20° C.

Material removed from each reactor appeared as a fine, dry, white powder. SEM imaging of the powder revealed discrete microparticles with semi-spherical shape and solid interiors (FIG. 10). The example demonstrates that solid core microparticles can be produced from a high-boiling point (202° C.), high melting point (−25° C.) solvent in the apparatus. Furthermore, this example demonstrates that NMP, a highly versatile solvent useful for dissolving a broad range of polymers and poorly soluble molecules, can be used in the apparatus to produce microparticles.

### EXAMPLE 9

This example demonstrates the preparation of microparticles containing approximately 10 wt % of a peptide using the procedure described in Example 2 and the apparatus of FIG. 3, FIG. 4 or FIG. 20. A formulation approach was utilized in which the peptide and polymer were co-dissolved in the polymer solvent. A 0.11 g/ml solution of a 50:50 PLGA polymer (5050 DL2A, Lakeshore Biomaterials) in N-methylpyrrolidone (NMP) was prepared by dissolving 585 mg of polymer. In a separate vial, 75.9 mg of the peptide was weighed, and 152 mg of methanol added. The polymer solution was then added to the peptide solution to form a single formulation solution with a theoretical weight concentration of 0.105 mg solids/mg solution.

140 ml of 75:25 v/v Pentane:Ethanol non-solvent mixture was added to the collection reservoir at room temperature. The non-solvent was then chilled and maintained at a target temperature of −115° C. The vaporized liquid nitrogen used in the cooling system was supplied at −150° C. Sparging of N₂ gas was initiated at ˜50 sccm.

Approximately 2 ml of the formulation solution was loaded into a syringe and spraying was initiated at a flow rate of 0.5 ml/min and an atomization power of 2.0 W using a Sono-Tek 25 kHz nozzle. Following atomization, the non-solvent to solvent ratio was ˜70:1 v/v; temperature was ramped to −5.0° C. over ˜70 minutes and equilibration for a total extraction time of 2 hours. Particles were filtered in place and rinsed with cold pentane (−80° C.) before drying under vacuum over ˜18 hours using a ramp cycle from −20° C. to 20° C.

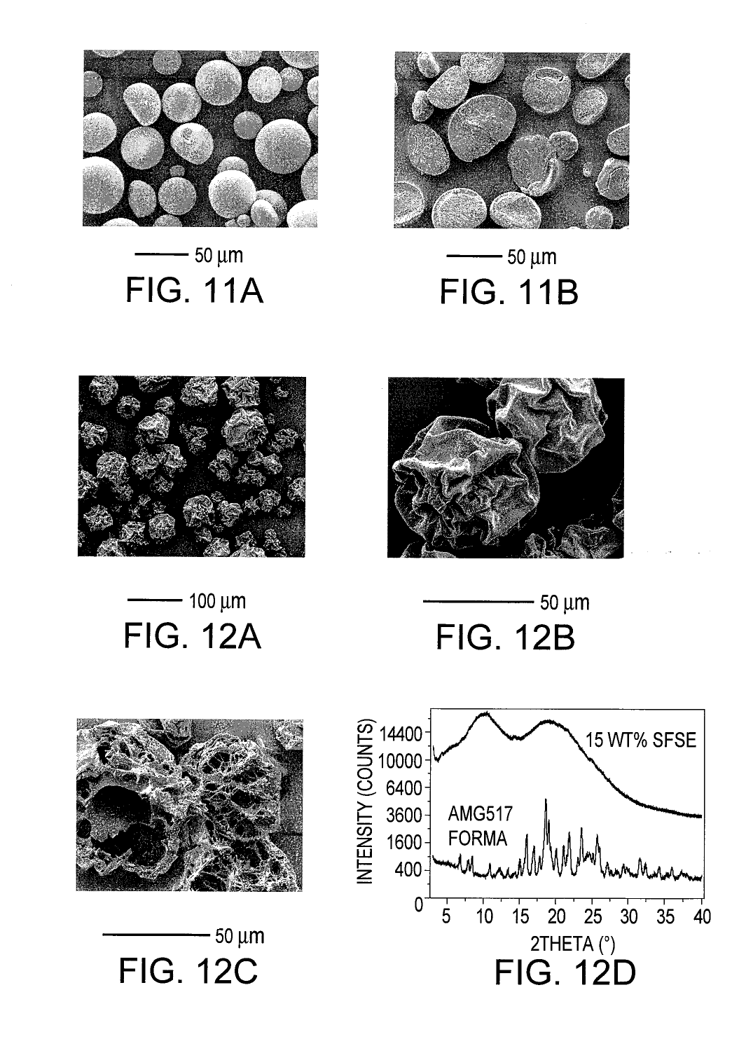

Material removed from each reactor appeared as a fine, dry, white powder. Recovered yield was 79%. SEM imaging of the powder revealed discrete microparticles with semi-spherical shape and solid interiors (FIG. 11). Determined peptide load in the dry particles was measured by HPLC following extraction recovery from glacial acetic acid and was 11 wt %. The example demonstrates that with appropriate selection of a non-solvent mixture, freezing temperature, and steady-state extraction temperature, solid core polymer microparticles containing an active agent can be produced from a high-boiling point (202° C.), high melting point (−25° C.) solvent in the apparatus. Furthermore, the active agent is encapsulated with high efficiency.

### EXAMPLE 10

This example demonstrates the preparation of the compound, N-(4-(6-(4-(trifluoromethyl)phenyl)pyrimidin-4-yloxy)benzo[d]thiazol-2-yl)acetamide, as an amorphous solid dispersion in hypromellose acetate succinate (HPMCAS) microparticles using spray freeze solvent extraction. For convenience, throughout this example, the term “Compound” refers to N-(4-(6-(4-(trifluoromethyl)phenyl)pyrimidin-4-yloxy)benzo[d]thiazol-2-yl)acetamide. HPMCAS and N-(4-(6-(4-(trifluoromethyl)phenyl)pyrimidin-4-yloxy)benzo[d]thiazol-2-yl)acetamide were screened for co-solubility in solvents for preparation of 15% and 50% Compound-loaded particles as shown in Table I. Solvent micro-droplets containing polymer/Compound were frozen in liquid nitrogen, and solvent extracted by a non-solvent to form particles. Compound load was determined by HPLC. Scanning electron microscopy, X-ray powder diffraction and thermo-gravimetric analysis were used to assess particle morphology, crystallinity, and residual solvent levels, respectively.

TABLE I
Solubility screening of HPMCAS and Compound
identified solvents for SFSE evaluation

| | HPMCAS | Compound | | |
|---|---|---|---|---|
| | Solubility @ | Solubility @ 25° C. | MP/BP Solvent | |
| Solvent | 100 mg/ml | (mg/ml) | (°C.) | |
| Pentane¹ | Insoluble | 0.0076 | −129.7 | 36.1 |
| Methanol | Soluble | 5.77 | −98.0 | 64.6 |
| Ethanol¹ | Insoluble | 4.56 | −114.1 | 78.3 |
| 1-Propanol | Insoluble | 4.29 | −126.0 | 97.2 |
| Dichloromethane | Insoluble (gel) | 14.9 | −96.7 | 39.8 |
| Chloroform | Insoluble (gel) | 32.3 | −63.7 | 61.7 |
| Dichloroethane | Insoluble (gel) | n/a | −35.3 | 83.5 |
| Toluene | Insoluble (gel) | 139.9 | −93.0 | 110.6 |
| NMP² | Soluble | >50.0 | −24.0 | 202.0 |
| Acetone² | Soluble | 67.1 | −94.3 | 56.2 |
| THF | Soluble | 78.7 | −108.4 | 66.0 |
| Ethyl Acetate² | Soluble | 30.2 | −83.6 | 77.1 |
| DMF | Soluble | n/a | −61.0 | 153.0 |
| DMAC | Soluble | n/a | −20.0 | 164.0 |
| Ethyl Lactate² | Soluble | ˜33 | −26.0 | 154.0 |

¹Non-Solvents - Pentane and Ethanol were non-solvents for the polymer and were poor solvents for the Compound. The Compound was least soluble in pentane.
²Polymer/Compound Solvents -Ethyl Acetate and Acetone are volatile solvents with low MPs; Ethyl Lactate and NMP are non-volatile solvents with higher MPs

Solvent-selection was based on process-specific criteria and considerations. For example, pair-wise solubility/miscibility between components required for SFSE. Solvents were chosen in which the Compound and polymer were both soluble and the non-solvent was selected based on the fact that the solvent was miscible with the anti-solvent but the polymer and Compound were insoluble in the antisolvent. For example, pentane was chosen over ethanol as the non-solvent because the Compound was less soluble in pentane. Further, solvents having a higher melting point were selected over lower melting point solvents. The miscibility of SFSE solvents in pentane and the temperature dependence of miscibility were considered. For example, ethyl lactate was chosen over N-methylpyrrolidone because of the solvent/non solvent miscibility requirement.

To prepare the particles, HPMCAS and Compound were co-dissolved in ethyl lactate for a dissolved solids concentration of 7.3 wt % and 5.5 wt % for the 15% and 50% Compound formulations, respectively. The solution was atomized at 0.5 ml/min into liquid nitrogen using a 25 KHz ultrasonic nozzle (Sono-Tek™) tuned to 3-4 Watts. Upon evaporation of the LN2, chilled pentane was added at −120° C. (125:1 Pentane:Ethyl Lactate v/v) and a motorized mixer was activated. Batch temperature warmed to −80° C. before transferring containers to an 18° C. bath until the batch temperature reached −10° C.; batches were then transferred to a bath at 0° C. and allowed to equilibrate over about 12 hours. Following extraction, particles were filtered and rinsed with cold pentane (−80° C.), and vacuum dried in-place using house vacuum for about 20 hours at room temperature.

Compound content within HPMCAS solid dispersion microparticles was determined using HPLC by standard methods. Encapsulation efficiency was defined as: [(Determined Wt % Compound)÷(Theoretical Wt % Compound)]×100%.

A thin layer of dry particles were placed on carbon backed adhesive SEM stubs, and sputter-coated with Au/Pd in preparation for scanning electron microscopy analysis. For freeze-fracture SEM, particles were sandwiched between two adhesive stubs, chilled in liquid nitrogen, and rapidly separated to produce two fracture surfaces for sputter-coating. Samples were imaged using a Philips XL30 SEM.

Mass-loss properties were characterized using thermo-gravimetric analysis in a TGA 2950 by TA Instruments. Samples of 5-10 mg were heated at 10° C./min over a temperature range of 25° C. to 300° C. Data analysis was with a thermal analyzer (Universal Analysis 2000, TA Instruments).

X-ray diffraction powder data (“XRPD”) was obtained using a Phillips automated x-ray powder diffractometer (X'Pert), equipped with a fixed slit.

A PW337310 LFF(1.54060 Å) CuKα X-ray tube was used with voltage and current of 45 kV and 40 mA, respectively. Samples of 5-10 mg were prepared on the sample holder and the stage rotated over the range of 20 from 30-40°. Normal scans of 10 minutes (low resolution) or 6 hours (high resolution) were collected.

SEM showed that discrete particles with ‘wrinkled’ spherical exterior structure and phase-inverted cores were made under these conditions. (See FIGS. 12a-c) For 15 wt % particles macroscopic sponge-like pores were visible on interior, indicative of phase inversion on formation. For 50 wt % particles some cores reveal ordered, crystal-like structure, indicating polymer/Compound de-mixing. The limited miscibility of ethyl lactate in pentane below −30° C. is thought to be the likely driver for the observed phase separation.

TGA was used to estimate residual solvent levels in particles as the total weight loss (TWL) before thermal degradation. For 15 wt % particles total weight loss was 2.4% and for the 50 wt % particles total weight loss was 11.5% indicating 2.4% and 11.5% residual solvent in each preparation, respectively. HPLC analysis indicated that 15 wt % particles had an encapsulation efficiency of about 77.3% and the 50 wt % particles had an encapsulation efficiency of 84.6%. A six hour XRPD scan of the microparticles is shown in FIG. 12D demonstrating that the 15 wt % particles were an amorphous powder.

### EXAMPLE 11

This example demonstrates a spray-freeze drying encapsulation process by direct atomization into a chilled extraction solvent (ACES), in the absence of a liquefied gas, as described in Example 2. Heat transfer models were developed to estimate droplet freezing times, t_{f} for the ACES process and for atomization into liquid nitrogen (ALN2). In ACES the model was used to identify operating conditions where solvent extraction, non-solvent influx, and droplet deformation where minimized atomization into liquid nitrogen (ALN2), as described in Example 1, a forced-convection heat transfer model was developed for spherical droplets falling through the headspace gas using an experimentally determined temperature gradient. For ACES, a one-dimensional heat-conduction model was developed to estimate t_{f} upon contact of a droplet with the liquid surface. Using two extraction solvents (n-pentane and iso-pentane) at various temperatures, and dichloromethane and dichloroethane as polymer solvents (MP −97° C. and −35° C. respectively), projected freezing times were varied. Impact on particle morphology (by SEM) and solvent residuals (by GC) was assessed and the results compared with those obtained with microparticles made by ALN2.

Scanning electron microscopy indicated spherical, solid-core particles were formed by ALN2. Calculated t_{f}'s for dichloromethane and dichloroethane droplets in ALN2 were 98 ms and 46 ms, respectively. This was substantially shorter than the calculated headspace residence time, indicating freezing occurs prior to impact with the cryogen bed for droplets less than 100 μm. Calculated freezing times for the ACES conditions studied ranged from 9-36 ms. The slowest t_{f}'s resulted in collapsed, asymmetric particles with phase-separated cores and high nonsolvent residuals (greater than 10%). Intermediate t_{f}'s produced spherical-cap particles with rough exteriors and a mixture of solid-core and phase-separated structures. The shortest t_{f}'s produced smooth, spherical-cap particles with solid cores, closely resembling particles made by ALN2; further, residual solvents were similar or superior to those observed with ALN2. Phase separation within droplets, induced upon contact with the extraction solvent in the ACES system, was minimized for cases where t_{f}<12 ms, corresponding to a Stefan Number, Ste>1.3. These results were obtained with cryogen temperatures as high as −122° C.

In the present work heat transfer models were developed and droplet freezing times calculated for the ALN2 and ACES processes under various conditions. Blank microparticles were fabricated of 10 kD poly(D,L-lactide-co-glycolide) using dichloromethane and dichloroethane as polymer solvents, and pentane (both n- and i-) for extraction. Particle size and morphology were assessed, and residual solvent levels determined. Temperature conditions for ACES were selected initially to maximize freezing rate, based on the theoretical model. Freezing time scaled according to the dimensionless Stefan number (Ste), which captures the relevant process temperatures and solvent thermodynamic properties. The impact of freezing time on morphology was assessed across various solvent/nonsolvent pairs and temperatures according to the Stefan number, and an optimal ACES process space identified.

PLGA (5050 DL2A, M_{w}=10 kDa, Lot 4071-650) was obtained from Lakeshore Biomaterials, Inc. (Birmingham, Ala.). 1,2-Dichloroethane was obtained from EMD Chemicals (Gibbstown, N.J.). Dichloromethane, iso-pentane, and 10 N NaOH were obtained from Mallinckrodt Baker Inc. (Phillipsburg, N.J.). Methanol, n-pentane, DMSO, and HPLC-grade water were obtained from Honeywell Burdick & Jackson (Muskegon, Mich.). All solvents were of analytical grade.

For ALN2, PLGA (5050 DL2A) was dissolved in dichloromethane or dichloroethane at a concentration of 0.11 g/ml. The solution was atomized at 0.5 ml/min into a vessel containing liquid nitrogen using a 25 KHz ultrasonic nozzle (SonoTek Corp.; Milton, N.Y.) tuned to 1.4 W; a custom-built, closed spray chamber was used for containing the spray and creating a fixed cold vapor zone above the LN2 (FIG. 1). Following atomization, LN2 was allowed to evaporate to near completeness and chilled pentane was added at −120° C. (˜70:1 Nonsolvent:Solvent v/v) to begin extraction, while a motorized mixer was activated to stir the vessel contents. System temperature warmed to −85° C. before transferring containers to a cryogenic bath at either −30° C. (dichloroethane) or −83° C. (dichloromethane) for equilibration over ˜18 hours. Following extraction in n-pentane, particles were filtered and rinsed with cold pentane (−80° C.), before filter cakes were placed in a shelf lyophilizer (Virtis) pre-chilled to −40° C. Drying occurred under vacuum over 36 hours using a ramp cycle from −40° C. to 20° C.

In the ALN2 process (FIG. 1, FIG. 2), the spray-freeze chamber was designed to utilize liquid nitrogen as the cryogen (T_{c}=−196° C.). In the closed-chamber design, vacuum ports are located above the cryogen liquid surface, and below the atomization nozzle tip. As a result, a cold vapor zone, resulting from the boil-off of the liquid nitrogen, is created between the cryogen surface and the centerline position of the vacuum ports. The level of LN2 in the vessel is controlled throughout the atomization process. Above the vacuum ports, the nozzle tip is positioned within a relatively warmer gaseous region. The nitrogen boil-off rate was determined based on the measured fill rate required to maintain a constant liquid level.

For ACES, polymer solutions were prepared and atomized as above. A custom-built reactor was used for containing the spray and for controlling the temperature of the extraction nonsolvent throughout the freezing and extraction process (FIG. 3A, FIG. 4, FIG. 20). Cryogenically refrigerated coolant was used to maintain the temperature of the nonsolvent in the reactor. Nitrogen gas sparging at about 50 cm³/min was applied within the reactor during freezing and extraction to homogenize the reactor contents. Following atomization, the nonsolvent to solvent ratio was about 70:1 v/v. For all systems, temperature was ramped to −30° C. over about 45 minutes before equilibration for a minimum of 4 hours, unless noted. Particles were filtered in place and rinsed with cold pentane (−80° C.) before drying under vacuum over ˜18 hours using a ramp cycle from −30° C. to 20° C.

For SEM analysis, a thin layer of dry particles were placed on carbon-backed adhesive SEM stubs, and sputter-coated with Au/Pd. For freeze-fracture SEM, particles were sandwiched between two adhesive stubs, chilled in liquid nitrogen, and rapidly separated to produce two fracture surfaces for sputter-coating. Samples were imaged using a Philips XL30 SEM.

For residual solvent content (dichloroethane, dichloromethane, n-pentane, iso-pentane) was determined using a headspace gas chromatography method. 5-10 mg of microparticles (n=3) were carefully weighed, dissolved into either DMSO (dichloroethane samples) or 0.5N NaOH (dichloromethane samples) containing 50 μl methanol, and analyzed on a Hewlett Packard HP-6890 GC system. The system was equipped with a G1888A Headspace Sampler, HP Chemstation 3365 software, and a Chrompack™ Fused Silica Q-HT Capillary Column (CP-PoraPlot Q-HT™; 27.5 m×0.53 mm×20 mm). Detection was by flame ionization. The flow-rates were 40 ml/min for hydrogen, and 450 ml/min for air, and 35 ml/min for the carrier gas (helium), with a split ratio of 0.247:1. The headspace over temperature was 90° C., the column oven temperature was 220° C., and the detector temperature was 250° C. Injection volume was 1 ml. Vials and caps were acquired from Agilent Technologies. Standards were prepared for quantifying dichloroethane, dichloromethane, n-pentane and iso-pentane via a serial dilution procedure using methanol as a carrier solvent. 50 μl of each standard was added to DMSO (dichloroethane samples) or 0.5N NaOH (dichloromethane samples) and analyzed as above.

Particle size was measured using a Malvern Mastersizer 2000 equipped with a Hydro 2000 μP wet dispersion cell (Malvern Instruments Ltd; Worcestershire, UK). Approximately 10 mg particles were dispersed in 0.5 ml Sedisperse A-12 (Micromeritics Corp.; Norcross, Ga.), and added to the dispersion cell containing hexane. Particle size distribution was calculated using the Fraunhoffer optical diffraction model to yield the volume based diameter parameters at 10%, 50%, and 90% cumulative volume percent: d_{0.1}, d_{0.5}, and d_{0.9}, respectively.

Surface tension of dichloroethane and a 0.11 g/ml solution of 5050 DL2A polymer in dichloroethane was measured using a Krüss K100 tensiometer (Krüss gmbH; Hamburg Germany). The Wilhelmy Plate method was used (Platinum plate). 100-ml of liquid was added to a clean 70 mm sample vessel and equilibrated at 25.0° C. prior to measurement.

Polymer solution viscosity was determined using a Gilmont® falling ball viscometer (Model GV-2100; Cole-Parmer). A stainless steel ball (density 8.02 g/ml) was used. A 0.11 g/ml solution of 5050 DL2A polymer (Lot 4071-650; Lakeshore Biomaterials Inc) was prepared in dichloroethane and added at room temperature (˜20° C.) to the viscometer. The time of descent for the ball was measured three times. The viscometer constant (K=0.2) was determined using N-methylpyrrolidone (viscosity 1.65 cp) as a reference.

ALN2 headspace temperature as a function of axial distance in the chamber was determined under atomizing conditions with dichloromethane. A type K thermocouple probe (Newport Electronics Inc.; Santa Ana, Calif.) was marked in 5 mm increments and inserted vertically through a port at the top of the atomization chamber (FIG. 1) with a radial distance approximately 40 mm off-center of the ultrasonic nozzle. Temperatures were recorded using a thermocouple data-logger (Model CP-92000; Cole-Parmer), over several periods of the liquid nitrogen re-filling and vaporizing. The average and standard deviation of the measurements were computed at each probe position. The ACES headspace temperature profile as a function of axial distance in the chamber was determined under non-atomizing conditions by removing the nozzle and inserting the thermocouple probe through the nozzle port into the reactor headspace above the liquid (FIG. 3A, FIG. 4). Pentane in the reactor was chilled to −122° C., and the temperature recorded at each probe position after stabilizing for several minutes.

Encapsulation by SFSE involves atomization, droplet cascade through a headspace, and impact of droplets with the cryogen bed (Table II). Each of these steps may entail a number of various events and contributing factors, outlined in Table II. Of particular note are events likely to differ between the ALN2 and ACES process. In the former, due to the tremendous temperature gradient, substantial droplet cooling and even freezing may occur in the headspace. In the latter, if freezing is not sufficiently rapid when droplets contact the cold non-solvent surface, droplet spreading or deformation may occur, as well as polymer solvent extraction and nonsolvent (cryogen) influx. Each of these possible events depends on a variety of experimental factors (Table II).

TABLE II
Factors influencing SFSE process steps

| Step | Possible Events¹ | Factors |
|---|---|---|
| Atomization | Transient heating | Nozzle type |
| | Droplet propulsion | |
| Droplet fall | Solvent evaporation | Solvent vapor pressure, |
| through headspace | Droplet cooling, | heat capacity, thermal |
| | supercooling, or | conductivity |
| | freezing | Temperature gradient in |
| | | headspace |
| | | Headspace cooling |
| | | capacity |
| | | Droplet size |
| | | Droplet velocity |
| Droplet impact | Droplet quench | Cryogen temperature |
| with cryogen | freezing | Droplet freezing rate |
| | Droplet spreading, | Miscibility of polymer |
| | deformation | solvent and cryogen (or |
| | Solvent extraction | extraction solvent) |
| | by cryogen | |
| | Nonsolvent influx | |

¹Events likely to differ between ALN2 and ACES processes are indicated in italics.

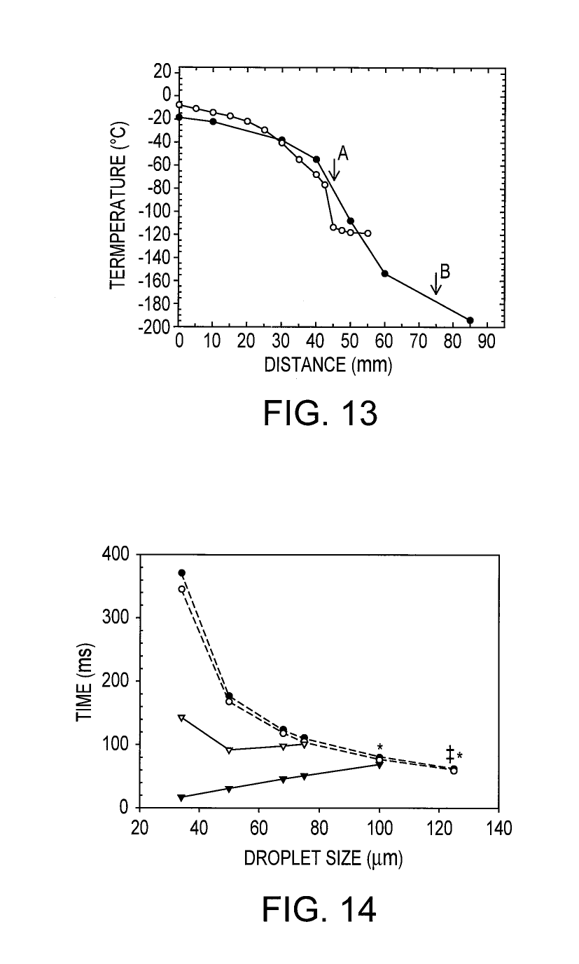

Headspace conditions were characterized by monitoring temperature as a function of distance from nozzle tip. The temperature profile in the top 45 mm of headspace is remarkably similar for the ALN2 and ACES process vessels (FIG. 13). For ACES this height corresponded to the cryogen fill level, while in ALN2 it corresponded to the position of the vacuum ports placed approximately 30 mm above the cryogen level (FIG. 1, FIG. 2). This design was required to prevent freezing at the nozzle tip and resulted in a steep temperature gradient below the ports (FIG. 11), which extended below the freezing points of both polymer solvents used in this study (dichloromethane, MP −96.7° C.; dichloroethane, MP −35.3° C.).

Gas flows, and consequent cooling capacities, differed significantly between the two headspace regions. For ALN2, cryogen lost to boiling was estimated at 2.6 g/s, corresponding to a cooling capacity of −58 J/s and far exceeding the −2.4 J/s required to freeze dichloromethane at the atomization rates studied. In contrast, the cooling capacity of the sparge gas in ACES was −0.14 J/s, considered inconsequential.

Based on the experimentally determined headspace temperature profiles (FIG. 13) and the gas flow cooling capacities, the heat transfer model developed for ALN2 focused on assessing freezing rate in the headspace while that for ACES considered direct contact with the nonsolvent phase as the predominant heat transfer interface. Cryogen nonsolvents used in the ACES process were n-pentane (MP −129° C.) and iso-pentane (MP −159° C.). Both have appreciable miscibility with, and freezing points substantially below, the polymer solvents used in the study.

Droplet size distributions, assumed to be identical for the atomization nozzles utilized, were estimated from the determined sizes of fabricated particles shown by SEM to be non-porous, the known polymer density (1.3 g/mL), and the initial polymer concentration (0.11 g/mL). The calculated range of 34-125 μm (d_{0.1}-d_{0.9}), with a median of 68 μm, was consistent with specifications provided by the nozzle manufacturer. Unless otherwise stated model calculations were performed using the median size for both processes; solvent evaporation prior to freezing was ignored, due to the presumed rapid saturation of the headspace on initiation of atomization.

Droplet freezing in the ALN2 process was assessed by estimating the time required for freezing, t_{f}, based on the temperature profile in FIG. 13 and the estimated residence time in the headspace, tᵣₑₛ. Droplets for which t_{f}<tᵣₑₛ were concluded to have frozen before contacting the cryogen. Calculations were performed for a range of droplet sizes encompassing d_{0.1}-d_{0.9}. We assumed polymer solvent properties were temperature invariant in the region of interest (Table A.1). Nitrogen gas counter-flow, estimated to be less than 0.02 m/s, was concluded to contribute negligibly to droplet terminal velocity and thus ignored. We further assumed that at each temperature the cold vapor constituted an infinite heat sink. Calculations focused on the 30 mm directly above the cryogen bed, assuming droplets were at room temperature on entering this region; the impact of this condition is discussed below.

Droplet terminal velocity vₜ is a function of its diameter Dₚ and density ρₚ, and the density (ρ_{f}) and viscosity (μ_{f}) of the nitrogen headspace medium. Reynolds numbers ranged from 2.4 to 9.4 over the temperature region of interest. For intermediate Re,

$${v}_{t} \approx {[ \frac{2 g}{27} ( \frac{{ρ}_{p}}{{ρ}_{f}} - 1 ) ]}^{5 / 7} {{D}_{p}^{8 / 7} ( \frac{{ρ}_{f}}{{μ}_{f}} )}^{3 / 7} . \qquad ( 1 )$$

corresponding to residence times of 60-350 ms for the expected distribution of dichloromethane droplet sizes; for the median droplet size, tᵣₑₛ=120 ms (FIG. 14). Similar results were obtained for dichloroethane. Residence time is inversely related to droplet size; for larger droplets, the terminal velocity is larger and the residence time is shorter.

A forced-convection cooling model can be used to estimate t_{f}. The mean heat transfer coefficient hₘ for a sphere traveling in an infinite fluid at velocity Vₚ is

$$\frac{{h}_{m} {D}_{p}}{{k}_{f}} = 2.0 + 0.60 {( \frac{{D}_{P} {V}_{P} {ρ}_{f}}{{μ}_{f}} )}^{1 / 2} {( \frac{{C}_{p , f} {μ}_{f}}{{k}_{f}} )}^{1 / 3} , \qquad ( 2 )$$

where Vₚ the terminal velocity, k_{f} is the thermal conductivity and C_{p,f} is the heat capacity of the nitrogen gas. The heat flow at the interface is thus:
Q=−h^{m}A·(Tₛ−T_{c}) (3)
where A is the interfacial surface area of the particle, Tₛ is the particle surface temperature, T_{c} is the cryogen temperature (equal to the headspace gas temperature; FIG. 13), and Q is the rate of heat removal.

Upon calculating hₘ, the heat transfer coefficient at the thermal boundary between the droplet and the cold fluid, the value of the Biot number, Bi=(hₘ Dₚ/2 kₚ) may be obtained. The Biot number (Bi) is a dimensionless number relating the convective heat flow at the interface to the conductive heat flow within the particle and given by
Bi=hₘDₚ/2kₚ, (4)
where kₚ is the droplet thermal conductivity. Determining hₘ from (2), Bi was determined to range from 0.10-0.23 for all droplet sizes and headspace temperatures considered. Since Bi <<1, cooling of the droplet is convection-limited and temperature gradients across the radial dimension of the droplets can be neglected. For convection-limited heat transfer in a sphere, the cooling rate is:

$$\frac{dT}{dt} \approx \frac{- 6}{{D}_{P}} {h}_{m} ( {T}_{0} - {T}_{c} ) ( \frac{1}{{ρ}_{P} {C}_{p , P}} ) , \qquad ( 5 )$$

with T₀ as the uniform particle temperature.

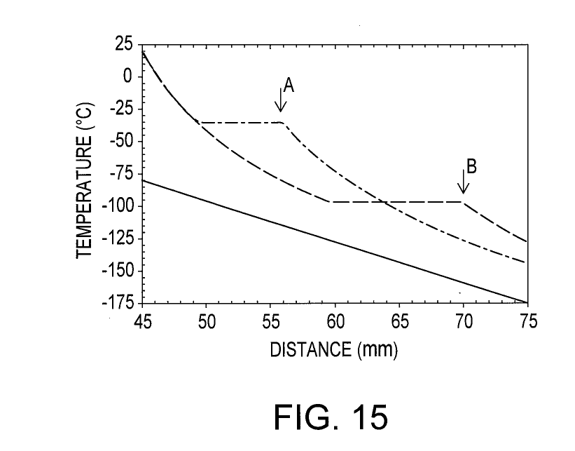

Freezing time for a droplet in the cold vapor layer consists of two parts—the time for the droplet to cool from its initial temperature to the solvent melting point (Tₘₚ), plus the time for the particle to lose its latent heat of fusion (L_{fus}) at particle temperature T_{0=T}ₘₚ. To estimate the freezing time, t_{f}, a step-wise integration was performed for each Dₚ in FIG. 14 with Δt=0.001 sec, T_{c,i}=−80° C. and T_{0,i}=20° C. A linear fit of the experimentally observed headspace temperature gradient was used (FIG. 15). Instantaneous velocity of the particle was calculated from the temperature-dependent properties of nitrogen using equation (1). Equation (2) was used to calculate the average heat transfer coefficient, and equation (4) was used to calculate the instantaneous cooling rate. Based on these values, the new position (Z), T_{c} and T₀ were calculated. Constant properties of the polymer solvent were assumed. Once the particle temperature reaches Tₘₚ, equation (3) was used to calculate the rate of latent heat removal from the particle at constant T₀=Tₘₚ, assuming similar properties of liquid and solid polymer solvent. Droplet temperature after freezing was similarly determined using equation (4).

Freezing times determined for droplets of the size range of interest appear in FIG. 14. For dichloroethane, freezing time decreases with droplet size because the entire headspace region is below the solvent freezing point; for all but the largest particles (where residence time is the shortest) t_{f}<tᵣₑₛ. The temperature difference between solvent freezing point and headspace vapor is much smaller in the case of dichloromethane, and is positive in the top quarter of the region. Further, (T₀−T_{c}) becomes small before reaching the freezing point, resulting in lower cooling rate (equation (4)). As a result, freezing occurs more slowly for the smallest droplets than for those of moderate size, in contrast with dichloroethane. However, as with dichloroethane, for all but the largest particles, t_{f}<tᵣₑₛ. The distance-dependence of the temperature of median-sized dichloroethane and dichloromethane droplets in the ALN2 headspace is depicted in FIG. 15.

The impact of model assumptions regarding solvent evaporation and initial droplet temperature at the top of the cold vapor zone was assessed. For dichloromethane, a 50% loss of solvent due to evaporation will result in droplet shrinkage (for example, from 100 to 80 μm), but this is not sufficient to cause a change in the predicted outcome of impact with the cryogen bed prior to completion of freezing (FIG. 14). A 100 μm dichloromethane droplet entering the vapor layer at −80° C. (instead of 25° C.) will reach the freezing point in 42 ms (about 30 ms faster than room temperature droplets); regardless, cryogen bed impact is predicted at 76 ms, prior to completion of freezing. We conclude that in the ALN2 process all but the largest droplets are expected to freeze prior to contact with the cryogen bed for both polymer solvents studied.

In the ACES process, droplets are likely to impact the nonsolvent liquid surface while still in a liquid state. Droplet impaction with solid surfaces has been characterized both theoretically and experimentally treated by several research groups. In contrast, droplet impaction with liquids which has received little attention. Droplets impacting on a solid surface will spread, deform, and recoil. Functional relationships based on kinetic energy of the impacting droplet, surface energy of the spreading/deformed droplet, and viscous work of flow of the droplet describe these events. In molten drop deposition onto a cold surface, the fluid dynamics model for spreading, deformation, and recoil are coupled with a heat transfer model based on thermal conduction and convection within the droplet and the heat conduction across the solid-liquid interface. Propagation of a freezing front from the contact area of the spreading droplet must also be considered. These expressions can be numerically solved to model the shape of a solidified particle and have been used to derive a set of dimensionless similarity parameters that can be used to empirically study the systems of interest. The Weber number We scales the driving force for spreading as the ratio of the kinetic energy of the impacting droplet to the capillary force imbalance at the contact line between the solid surface, liquid droplet, and gas (air):

$$We = \frac{{ρ}_{P} {V}_{P}^{2} {R}_{P}}{σ} = \frac{{ρ}_{P} {V}_{P}^{2} {D}_{P}}{2 σ} \qquad ( 6 )$$

where ρₚ is density of the droplet, Vₚ is the droplet velocity, Rₚ is the droplet radius, and σ is ordinary surface tension of the liquid droplet. When the kinetic energy of impaction is small compared to the capillary force imbalance, We is less than 1, spreading is driven by capillary forces. Such is the case for droplets of small size, low density, and low velocity. Molten droplets deposited at low We assume a ‘spherical cap’ shape upon solidification.

In equation 6 We was determined for ACES using the droplet size and velocity values discussed above, pure solvent values for density, and experimentally determined surface tensions for dichloroethane and dichloromethane. The ordinary surface tension of the dichloroethane polymer solution was 32.5 mN/m, comparable to that determined for pure solvent (32.0 mN/m). As shown in Table III, We ranged from 0.003 to 0.552 for the droplet size range of interest. A model for molten droplet deposition and solidification at low We was therefore used to analyze ACES. (Spherical-cap shapes were observed by SEM to result from some ACES process conditions, further supporting this approach; see below.)

TABLE III
Parameters calculated for the droplet impaction model¹

| | | | | t_{spread} | t_{osc} | t_{visc} |
|---|---|---|---|---|---|---|
| D_{P} (μm) | V_{P} (m/s)² | We | Oh³ | (ms) | (ms) | (ms) |
| 34 | 0.07 | 0.003 | 0.09 | 0.01 | 0.01 | 0.2 |
| 68 | 0.24 | 0.075 | 0.06 | 0.04 | 0.04 | 0.6 |
| 125 | 0.48 | 0.552 | 0.05 | 0.1 | 0.1 | 2.0 |

¹Using dichloroethane as the solvent with physical properties measured at ambient temperature (25° C.).
²Terminal velocity computed for nitrogen gas at temperature of −80° C.
³Ohnesorge number computed using the measured viscosity of a 0.11 g/ml polymer solution at room temperature (2.3 cP).

The Ohnesorge number Oh scales the force that resists spreading (viscosity) relative to the ordinary surface tension within the droplet:

$$Oh = \frac{{μ}_{P}}{\sqrt{{ρ}_{P} σ {R}_{P}}} . \qquad ( 7 )$$

where μp is the viscosity, measured to be 2.3 cp. Table III summarizes the calculated values for Oh across the droplet size range of interest. At low We and Oh greater than 1, the droplet viscosity resists the spreading of the contact line, and during solidification of the droplet, arrest of the propagating contact line may occur. For Oh much less than 1, droplets will spread quickly, reach a maximum in the degree of spreading, and then begin to recoil; oscillations will ensue until either the viscous forces dampen the motion, or the molten droplet solidifies. For the dichloroethane system Oh much less than 1, indicating droplets spread quickly on the liquid surface. Spreading, oscillation, and viscous dampening times (t_{spread}, t_{osc}, t_{visc}) are given by the following:

$$\begin{matrix}{t}_{spread} \approx {t}_{osc} \approx \sqrt{\frac{{ρ}_{P} {R}_{P}^{3}}{σ}} , and & ( 8 ) \\ {t}_{visc} \approx \frac{{ρ}_{P} {R}_{P}^{2}}{{μ}_{P}} . & ( 9 )\end{matrix}$$

These expressions were applied to droplets of the size range of interest (neglecting viscous effects at the contact line, as the droplet cools); results appear in Table III and show t_{spread} and t_{osc} are one order of magnitude smaller than the t_{visc}, indicating rapid spreading followed by a limited number of droplet oscillations. Under these conditions very little of the droplet volume solidifies by the time spreading, oscillations, and viscous dampening are complete, and the bulk solidification time of the droplet can be estimated using a one-dimensional heat conduction model that decouples the fluid dynamics problem from the heat transfer model. Droplets are assumed to spread and then freeze. During spreading, local solidification at the contact line will determine the effective contact area for heat conduction during bulk solidification. Bulk droplet solidification time is as derived in the Appendix for Example 11:

$${t}_{f} = ( \frac{9 {D}_{P}^{2}}{32} ) ( \frac{{C}_{p , P} {ρ}_{P}}{{k}_{P}} ) \frac{{L}_{fus}}{{C}_{p , P} ( {T}_{mp} - {T}_{c} )} . \qquad ( 10 )$$

where C_{p,P} and kₚ are the heat capacity and thermal conductivity of the droplet, L_{fus} is the latent heat of fusion of the droplet, and Dₚ is the diameter of the freezing droplet. The grouped parameter:

$$Ste = \frac{{C}_{p , P} ( {T}_{mp} - {T}_{c} )}{{L}_{fus}} \qquad ( 11 )$$

is the Stefan number, which scales the driving force for heat flux to the latent heat of fusion for the droplet. The freezing time t_{f} is inversely proportional to Ste. Various ACES process conditions were identified which resulted in a range of calculated Ste, and consequent t_{f} ranging from 9 to 36 ms (Table IV). This range was of interest for several reasons. First, the process conditions identified include multiple solvent/nonsolvent pairs and cryogen temperatures. This served to test the hypothesis that freezing time is the most critical parameter in matching the ACES and ALN2 processes. Second, disparate process conditions in some cases resulted in similar calculated t_{f}'s, depending on the solvent/nonsolvent pairing. Third, all the calculated t_{f} values are similar to, and slightly lower than, those calculated for ALN2. While the timescales of solvent extraction and nonsolvent influx, competing events unique to ACES, are not known, the calculations suggest at least the possibility that an operating range might be identified where the two processes provided similar results.

PLGA microparticles were fabricated by ALN2 and ACES processes under the conditions listed in Table IV. In all cases microparticles were obtained with good yield (greater than 75%) and consistent particle size distribution; mean d_{0.1}, d_{0.5}, and d_{0.9} were 17, 31, and 55 μm respectively. Particle morphology was assessed by SEM. Residual polymer and extraction solvents were also assessed.

TABLE IV
Experimental conditions for ALN2 and ACES processes

| | | | | | Residual Solvent | |
|---|---|---|---|---|---|---|
| | | | | | (wt %) | |
| | | | | | | Non- |
| Process | Solvents | Ste | t_{f} (ms) ¹ | T_{c} (° C.) | Solvent | solvent |
| ALN2 | dichloroethane, | — | 98 | −196 | 0.06 | 1.8 |
| | n-pentane | | | | | |
| ALN2 | dichloroethane, | — | 46 | −196 | 0.7 | 3.0 |
| | n-pentane | | | | | |
| ACES | dichloromethane, | 0.4 | 36 | −122 | 0.11 | 12.6 |
| | n-pentane | | | | | |
| ACES | dichloroethane, | 0.4 | 34 | −65 | 4.4 ± | 11.7 ± |
| | n-pentane | | | | 0.4 | 0.6 |
| ACES | dichloromethane, | 0.9 | 16 | −152 | 0.07 | 6.0 |
| | i-pentane | | | | | |
| ACES | dichloroethane, | 0.9 | 17 | −95 | 1.3 | 2.8 |
| | n-pentane | | | | | |
| ACES | dichloroethane, | 1.3 | 12 | −122 | 0.05 | 1.1 |
| | n-pentane | | | | | |
| ACES | dichloroethane, | 1.8 | 9 | −152 | 0.13 | 0.1 |
| | i-pentane | | | | | |

¹ For median droplet size.

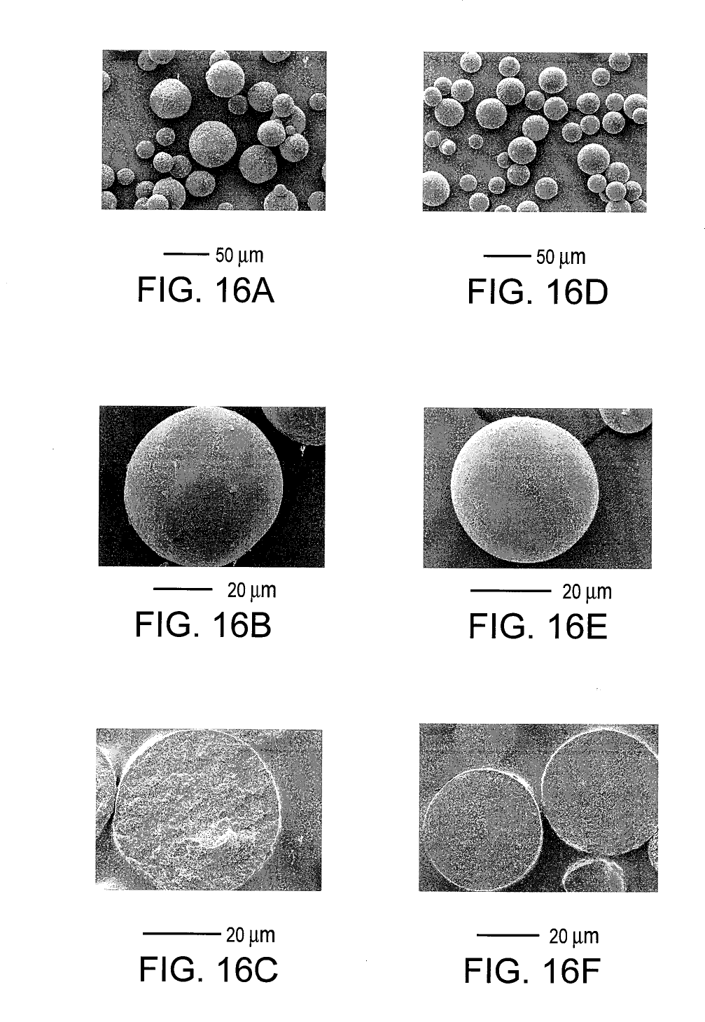

FIG. 16 shows the structure of PLGA microparticles produced by the ALN2 process using dichloromethane (FIGS. 16A-C) and dichloroethane (FIGS. 16D-E). For both polymer solvents, particles were discrete spherical particles with smooth exterior surfaces; cross fractures of the particles revealed homogeneous, solid internal cores and no discernable porosity.

As shown in Table IV, pentane residual levels observed with the ALN2 process were 1.8 wt % and 3 wt % for microparticles made with dichloromethane and dichloroethane, respectively. Dichloromethane residual solvent levels were 0.06%, about 10-fold lower than the dichloroethane level of 0.7 wt %. These values are consistent with literature reports. The mechanism of nonsolvent entrapment in SFSE processes has received less attention to date.

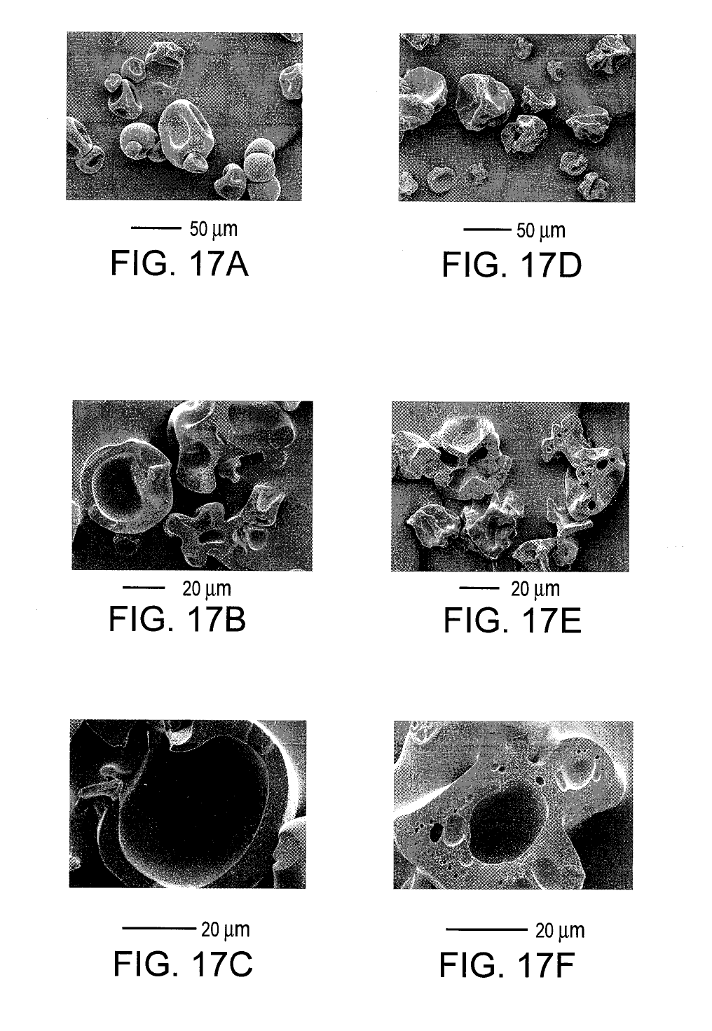

Microparticle lots made by the ACES process were grouped according to Ste and t_{f} (Table IV). Conditions expected to provide the longest freezing times are shown in FIG. 17. Two different sets of process conditions, dichloromethane with pentane at −122° C. and dichloroethane with pentane at −65° C. are depicted (FIGS. 17A-C and 17D-F, respectively), corresponding to Ste=0.4 and t_{f}=34-36 ms. In both cases discrete, collapsed particles are observed with hollow-core and collapsed-core internal structures characterized by a moderately thick polymer skin that collapses in toward the center of the particle. FIG. 17F reveals particles with collapsed-core internal structure and a polymer skin layer with larger, visible voids.

The morphologies observed in the ACES system for both dichloromethane and dichloroethane at Ste=0.4 suggest particle formation occurred via a phase-separation process, such as liquid-liquid mixing induced by nonsolvent influx. The ternary diagram for a polymer/solvent/nonsolvent mixture is divided into regions where the mixture may take the form of a rubbery or glass-like solid, a single phase liquid or gel, or a two-phase mixture of polymer-rich and solvent-rich domains. In general, phase separation within the two phase region of amorphous polymers such as PLGA always proceeds via liquid-liquid de-mixing into a polymer-rich liquid and a polymer-poor liquid. The thermodynamic properties of a three component polymer/solvent/nonsolvent system is strongly affected by the quality of the solvent and the solvent/nonsolvent interactions. However, from a kinetic aspect, nonsolvent uptake is slowed with higher polymer solution concentrations. Generally, liquid-liquid de-mixing does not play a large role for concentrated polymer solutions (greater than 20% w/w) during immersion precipitation. Instead, a gel is formed as solvent is removed from the organic phase, indicating that membrane morphology is largely determined by the type of phase transitions a system can undergo.

Two general classes of precipitation behavior are observed upon phase separation, depending on the thermodynamic and mass transfer characteristics of the system studied. Fast phase-inverting systems are characterized by rapid nonsolvent influx, leading to liquid-liquid de-mixing that was visible in the formation of macroscopic voids. Slow phase-inverting systems are characterized by slower nonsolvent influx, coupled with solvent extraction. These slow phase-inverting systems are observed to be less porous and more gel-like.

The morphology of the dichloroethane system at Ste=0.4 (FIGS. 17D-F) is consistent with a fast phase-inverting system, as the voids formed by the solvent rich domains are easily seen within the cross-fractured particles. Scanning electron micrograph imaging for the dichloromethane particles may indicate a slightly slower phase-inverting system, owing to the minimal porosity observed within the polymer skin layer. Nevertheless, the collapsed structure and macroscopic voids in the center of the both dichloromethane and dichloroethane particles are indicative of rapid phase separation on the surface of the droplet, resulting in a polymer skin.

Solvent trapping in phase-inversion processes is known to result from phase changes at the surface or within the forming particles. In phase-separation processes, both the dispersed polymer phase and the nonsolvent continuous phase are initially in liquid states, creating the opportunity for rapid exchange of solvent and nonsolvent across the droplet interface. Since the thermodynamic driving force for nonsolvent partitioning into the precipitated polymer phase is minimal, once liquid-liquid phase separation has occurred within the particle, domains rich in nonsolvent are believed to persist; solvent trapping often occurs. Typically, chlorinated solvents such as dichloromethane are present the particles at levels between 0.05% and 5% (depending on the polymer), while hardening agents such as hexane or heptane are present in the range of 2% to 5%. Nonsolvent residuals for the microparticles in FIG. 17 were extremely high (greater than 10%; Table IV), as was the residual dichloroethane level (4.4%); solvent trapping resulting from phase separation, and the observed polymer skin which could introduce a diffusion barrier during subsequent extraction and drying steps, may be responsible.

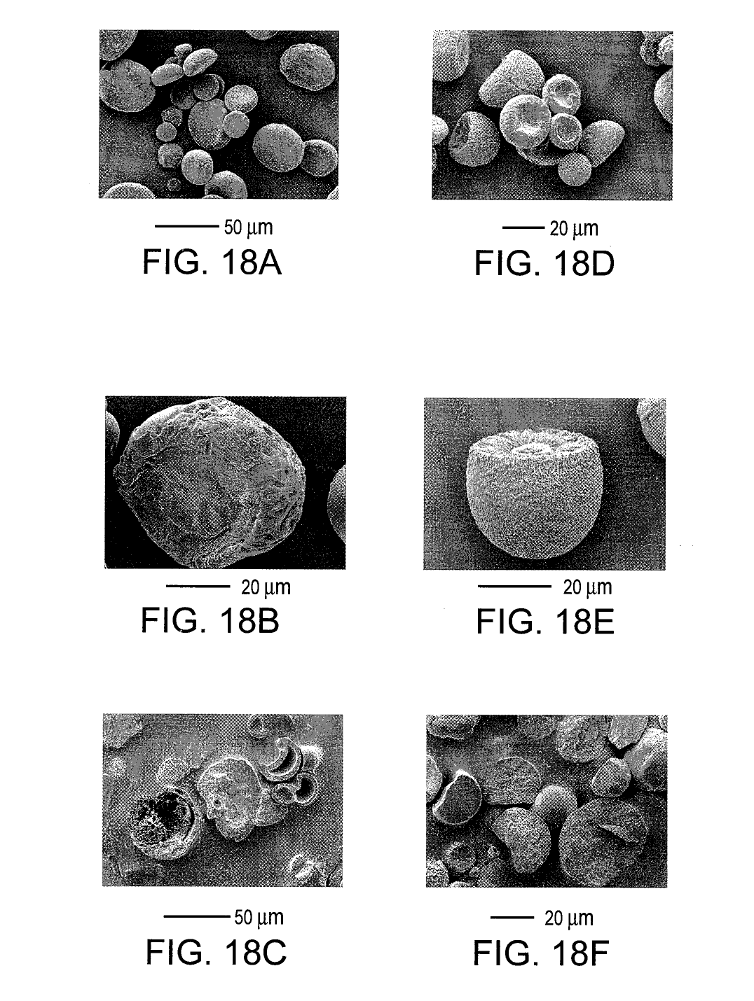

Particles prepared by ACES conditions providing intermediate freezing times are shown in FIG. 18. Two different sets of process conditions, dichloromethane with iso-pentane at −152° C. and dichloroethane with n-pentane at −95° C. were evaluated (FIGS. 18A-C and 18D-F, respectively) corresponding Ste=0.9 and t_{f}=16-17 ms. Particles were mostly discrete with spherical-cap external structures. Cross sections showed a mixture of solid-core, hollow-core and spongy-core internal structures. As in FIG. 17 the two process conditions providing intermediate t_{f}'s, despite their differences, resulted in remarkably similar morphologies.

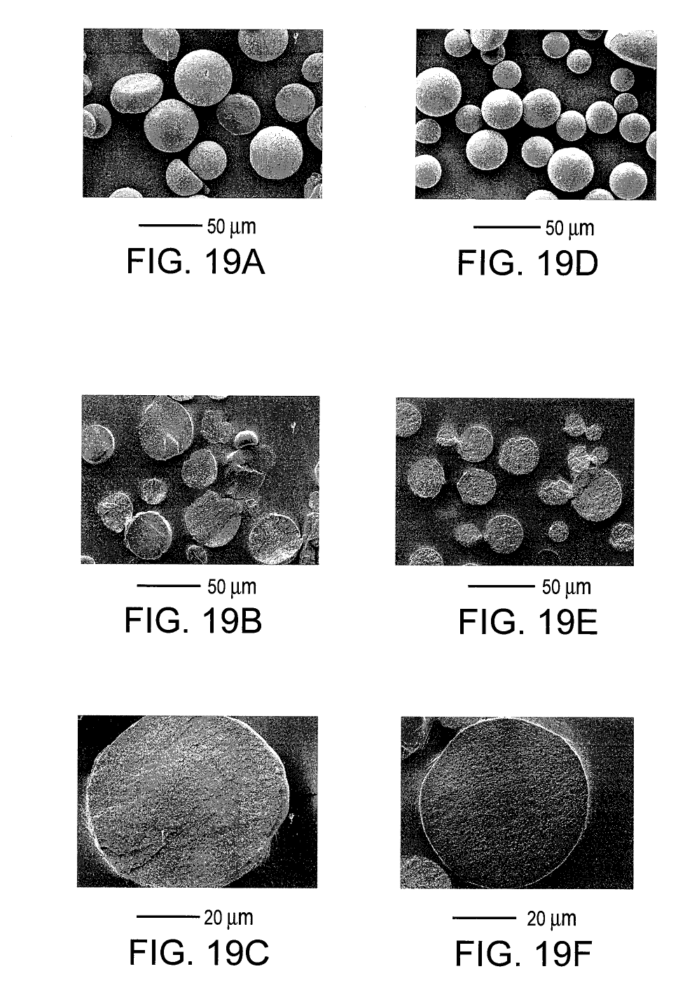

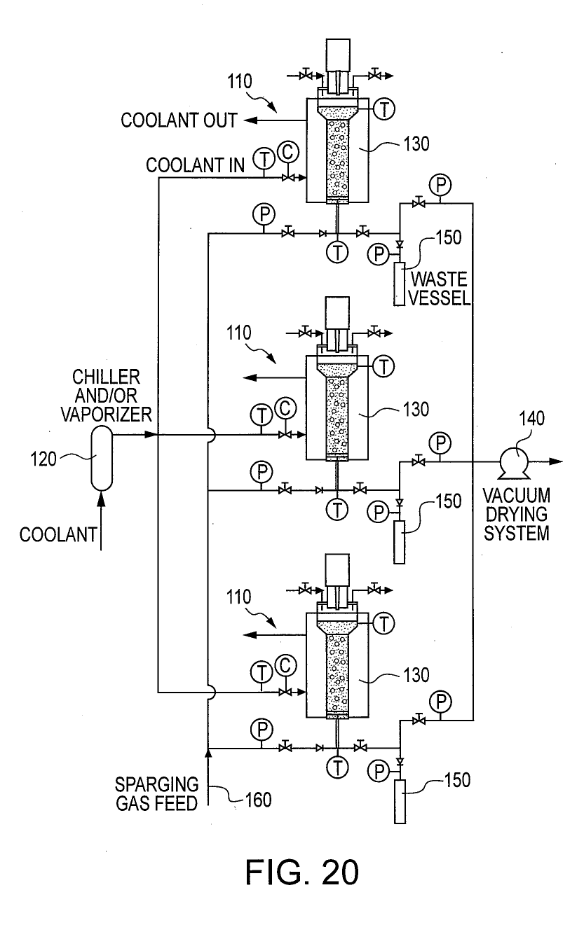

ACES conditions providing the most rapid freezing times are shown in FIG. 19. Two different sets of process conditions, dichloroethane with n-pentane at −122° C. and dichloroethane with iso-pentane at −152° C. were evaluated (FIGS. 19A-C and 19D-F, respectively) corresponding Ste=1.3 and 1.8, respectively, and t_{f}=9-12 ms. SEM indicated highly discrete, spherical-cap particles for both process conditions. Exterior surfaces were smooth, and freeze-fracture images (FIGS. 19C and 19F) indicated consistently homogeneous, solid internal structure with no observable porosity. The morphology is similar to the observed in particles prepared by ALN2 (FIG. 16). This finding suggests that the freezing rate exceeded rates for competing solvent extraction or nonsolvent influx for the values of Ste=1.3 and Ste=1.8.

Furthermore, the resulting spherical-cap shape of the particles and homogeneous, solid internal structure lend insight to the extraction process. The solid, homogeneous core suggests conditions under which solvent extraction occurred uniformly throughout the particle, likely while the droplet was in a frozen state. The degree of nonsolvent infiltration should also be minimal, as liquid-liquid de-mixing within the particle was likely avoided. The retention of a spherical-cap structure in the final product indicates the absence of significant plasticization of the polymer from the time of droplet impact to the end of solvent extraction.

Microparticles prepared with intermediate and rapid freezing times showed dramatically reduced residual solvent levels (Table IV). Under the best conditions, polymer solvent levels approximated those observed with ALN2. Nonsolvent residuals were substantially lower for ACES, especially in the case of dichloroethane with iso-pentane at −152° C., where the residual nonsolvent was 0.1%.

Droplet freezing times were estimated to be comparable for ALN2 and ACES processes, despite the different heat transfer mechanisms used. Process conditions identified through modeling defined an operating space, characterized by Ste greater than or equal to 1.3, where particle morphology and residual solvents were similar to those produced by ALN2 and the rate of freezing (calculated to be less than or equal to 12 ms) exceeded rates of extraction and nonsolvent influx as judged by morphology of the dry particles and residual solvent data. In contrast, the slowest ACES freezing rates resulted in high residual solvent levels and particle morphology consistent with a mechanism of particle formation involving phase separation.

Using dichloroethane as the polymer solvent and n-pentane as the extraction solvent, an operating temperature as high as −122° C. was possible. This is 74° C. higher than temperatures required in an ALN2 process, where operating temperature is dictated by the liquefied gas boiling point. The elimination of liquid nitrogen from the SFSE process has significant implications for scale-up requirements. The milder ACES process conditions identified can be achieved using cascade refrigeration equipment and custom refrigerant blends.

The ACES process under optimal conditions produced low residual solvent levels without the use of secondary drying. Secondary drying complicates downstream processing and exposes particles to conditions that may result in agglomeration or change in drug content. The remarkable decrease in residual solvent content observed under the best ACES conditions tested suggests that with further characterization of extraction conditions (for example, the time-temperature profile) the need for secondary drying may be eliminated altogether.

The following definitions and nomenclature are applicable in this example:

a characteristic length scale in 1-dimensional heat conduction model

A interfacial surface area of droplet (m²)

ACES atomization into a cold extraction solvent

ALN2 atomization into liquid nitrogen

Bi Biot number, Bi=(hₘ Dₚ/2 kₚ).

melt super-heat parameter

C_{p,f} heat capacity of nitrogen gas

C_{p,P} heat capacity of the droplet or particle solvent

Dₚ diameter of the droplet or particle, in meters

g gravity constant (9.81 m/s²)

hₘ heat transfer coefficient at the droplet/gas interface

k_{f} thermal conductivity of nitrogen gas

kₚ thermal conductivity of the droplet or particle solvent

L thickness of slab in the 1-dimensional heat conduction model

L_{fus} latent heat of fusion of droplet solvent, in J/kg

μ_{f} viscosity of the gaseous region above the liquid cryogen, in N*m

μₚ viscosity of the droplet solvent, in N*m

Oh Ohnesorge number

Q heat flow, in J/s

Re Reynolds Number

Rₚ radius of the droplet or particle, in meters

ρₚ density of the droplet or particle solvent, in kg/m³ or g/ml

ρ_{f} density of the gaseous region above the liquid cryogen, in kg/m³ or g/ml

σ ordinary surface tension of the droplet solvent, in N/m.

SFSE Spray Freeze/Solvent Extraction

Ste Stefan number

t_{f} characteristic freezing time of droplet in ACES model

t_{f} characteristic freezing time of droplet in ALN2 model

t_{osc} characteristic time for droplet recoil in the droplet impaction mode

tᵣₑₛ characteristic residence time of droplet in cold vapor region

t_{spread} characteristic time for droplet spreading in the droplet impaction model

t_{visc} characteristic time for viscous dampening in the droplet impaction model

T₀ initial temperature of the droplet, in ° C.

Tₘₚ melting point of the polymer solvent, in ° C.

T_{c} temperature of the cryogen for freezing the polymer solvent, in ° C.

Tₛ temperature of the droplet (surface) at the gas interface

Vₚ droplet velocity, in m/s

vₜ droplet terminal velocity, in m/s

We Weber number

TABLE A.1
Solvent properties used in freezing models.

| | MW | ρ_{P} * | T_{MP} | σ * | μ_{P} * | C_{P} | L_{fus} | k_{P} * |
|---|---|---|---|---|---|---|---|---|
| Solvent | (g/mol) | (g/ml) | (° C.) | (N/m) | (cP) | (J/kg*K) | (KJ/kg) | (W/m*K) |
| dichloromethane | 84.94 | 1.33 | −96.7 | 0.028 | 0.43 | 1204 | 72.5 | 0.139 |
| dichloroethane | 98.96 | 1.24 | −35.3 | 0.032 | 0.84 | 1364 | 89.3 | 0.143 |
| Pentane | 72.15 | 0.63 | −129.7 | 0.015 | 0.23 | 2293 | 116.4 | 0.113 |
| iso-pentane | 72.15 | 0.62 | −159.9 | 0.015 | 0.21 | 2285 | 71.1 | 0.110 |

* Temperature-dependent properties compiled at 20° C.

TABLE A.2
Properties of nitrogen gas

| Temperature | ρ_{f} | μ_{f} | C_{P, f} | k_{f} |
|---|---|---|---|---|
| (° C.) | (kg/m³) | (cP) | (J/kg*K) | (W/m*K) |
| −196 | 4.612 | 0.00544 | 1120 | 0.00750 |
| −175 | 3.563 | 0.00683 | 1070 | 0.00965 |
| −80 | 1.768 | 0.01257 | 1040 | 0.01815 |
| 25 | 1.145 | 0.01781 | 1040 | 0.02574 |

Compiled from NIST Web Book

A droplet impacting the liquid cryogen surface at low Weber number is assumed to first spread, forming a spherical-cap, and then freeze. The model for freezing assumes the spherical cap structure can be approximated as a cylindrical slab, such that heat transfer can be modeled as one-dimensional heat conduction through the slab. The one-dimensional heat conduction model to estimate freezing time is derived from the energy equation of change:

$$ρ \frac{D \overline{H}}{Dt} = - ∇ {q}_{z} , \qquad ( A .1 )$$

which is integrated over time (t) to give:

$${t}^{- 1} ( \frac{ρ}{k} ) Δ H = \frac{d}{dz} \frac{dT}{dz} . \qquad ( A .2 )$$

Integration over the axial dimension of the cylinder, z, gives:

$$\frac{ⅆ T}{ⅆ z} = {t}^{- 1} ( \frac{ρ}{k} ) Δ H \cdot z + {C}_{1} . \qquad ( A .3 )$$

For t→∞, dT/dz→0, thus C₁=0. Integrating a second time over z gives:

$$T = {t}^{- 1} ( \frac{ρ}{k} ) ( \frac{{z}^{2}}{2} ) Δ H + {C}_{2} . \qquad ( A .4 )$$

For z=0 (at the interface of the cryogen), T=T_{C}, thus C₂ T_{C}. Multiplying both sides by Cₚ and rearranging to express time, t, gives:

$$t = ( \frac{{z}^{2}}{2} ) ( \frac{{C}_{p} ρ}{k} ) \frac{Δ H}{{C}_{p} ( T - {T}_{c} )} . \qquad ( A .5 )$$

For a phase-change from liquid to solid, ΔH is the equivalent of the latent heat of fusion (L_{fus}). The temperature is constant at the point of freezing in this special case, thus T=Tₘₚ. Finally, the total time for freezing, t_{f}, is calculated using the maximum thickness of the slab, when z=L, giving:

$${t}_{f} = ( \frac{{L}^{2}}{2} ) ( \frac{{C}_{p} ρ}{k} ) \frac{{L}_{fus}}{{C}_{p} ( {T}_{m p} - {T}_{c} )} . \qquad ( A .6 )$$

For the spherical-cap structures observed in the ACES system, the characteristic particle size is similar to those produced in the ALN2 process. If we assume the diameter of a spherical droplet, Dₚ, is roughly equivalent to the diameter of the contact face of the spherical cap particle, then equating volumes for a spherical droplet and a cylinder with diameter D_{p gives:}

$$\frac{4}{3} {π ( \frac{{D}_{p}}{2} )}^{3} = {π ( \frac{{D}_{p}}{2} )}^{2} L \\ or \\ L = \frac{4}{6} {D}_{P} . \qquad ( A .7 )$$

Examination of a typical particle indicates the length L is approximately 0.75 Dₚ for the spherical-cap particle shown; substituting into equation A.6 gives the approximation for freezing time in terms of the droplet diameter and liquid properties:

$${t}_{f} = ( \frac{9 {D}_{P}^{2}}{32} ) ( \frac{{C}_{p , P} {ρ}_{P}}{{k}_{P}} ) \frac{{L}_{fus}}{{C}_{p , P} ( {T}_{m p} - {T}_{c} )} \qquad ( A .8 )$$

Several assumptions underlie the estimate for t_{f}: (1) heat transfer is by one-dimensional heat conduction at the interface between the cold solid surface and the spreading droplet; (2) the droplet is at its melting point, Tₘₚ; (3) the surface is isothermal at a temperature of T_{c}; (4) there is minimal contact resistance at the interface; (5) parameter values for C_{p,P}, kₚ, and ρₚ are constant and independent of temperature or the solid or liquid state; (6) the melting point of the droplet and its latent heat of fusion may be approximated as the melting point and latent heat of the pure solvent, with minimal change due to dissolved components. Under these conditions, the latent heat removed from the droplet during freezing is balanced against the one-dimensional heat flux across the contact interface to estimate t_{f}.

The estimate for bulk solidification time by one-dimensional heat conduction is valid so long as the latent heat must dominate over the effect of the droplet's superheat relative to its melting point (In β<Ste⁻¹), where β is the melt super-heat parameter:

$$β = \frac{( {T}_{0} - {T}_{m p} )}{( {T}_{m p} - {T}_{c} )} . \qquad ( A .9 )$$

This provision is satisfied for all conditions explored in this example.

It should be understood that various changes and modifications to the disclosed embodiments will be apparent to those skilled in the art. Such changes and modifications can be made without departing from the spirit and scope of the present subject matter. It is therefore intended that such changes and modifications be covered by the appended claims.

The invention is claimed as follows:

1. An apparatus for preparing particles comprising, an enclosed spray chamber that includes a spray nozzle and a collection reservoir, wherein the spray chamber has a gas inlet and a gas outlet and the spray nozzle is located in a gas layer at about or above the level of the gas outlet and the collection reservoir is located below the level of the gas outlet.

2. The apparatus of claim 1 further comprising a plurality of enclosed spray chambers that include a spray nozzle and a collection reservoir, wherein the spray chambers have a gas inlet and a gas outlet and the spray nozzle is located in a gas layer at about or above the level of the gas outlet and the collection reservoir is located below the level of the gas outlet.

3. The apparatus of claim 1 further comprising a plurality of enclosed spray chambers that include a spray nozzle and a collection reservoir, wherein the spray chambers have a gas inlet and a gas outlet and the spray nozzle is located in a gas layer at about or above the level of the gas outlet and the collection reservoir is located below the level of the gas outlet and wherein the spray chambers are joined to a common utility system.

4. The apparatus of claim 1, wherein the gas inlet is above the level of the gas outlet.

5. The apparatus of claim 1, further comprising a second gas inlet.

6. The apparatus of claim 1, wherein the gas inlet is located in the collection reservoir.

7. The apparatus of claim 1, wherein the gas inlet is a gas sparger located in the collection reservoir.

8. The apparatus of claim 1, wherein the collection reservoir contains a collection fluid.

9. The apparatus of claim 1, wherein the collection reservoir contains a collection fluid and particles.

10. The apparatus of claim 1, wherein the collection reservoir contains a collection fluid comprising a liquefied gas.

11. The apparatus of claim 1, wherein the collection reservoir contains a collection fluid comprising an anti-solvent.

12. The apparatus of claim 1, wherein the collection reservoir contains solid particles and a collection fluid comprising an anti-solvent.

13. The apparatus of claim 1, wherein a feed tube for collection fluid extends into the spray chamber.

14. The apparatus of claim 1, wherein a feed tube for collection fluid extends into the spray chamber and into the collection reservoir.

15. The apparatus of claim 1, wherein the outlet port leads to a vacuum source.

16. The apparatus of claim 1, further comprising at least two gas layers having distinct temperature profiles.

17. The apparatus of claim 1, further comprising at least two gas layers having distinct temperature profiles wherein a first gas layer is above a second gas layer.

18. The apparatus of claim 1, further comprising at least two gas layers having distinct temperature profiles wherein a first gas layer is above a second gas layer and the border between the first and second gas layers is near the level of the gas outlet port.

19. The apparatus of claim 1, further comprising at least two gas layers having distinct temperature profiles wherein a first gas layer is above a second gas layer and the border between the first and second gas layers is near the level of the gas outlet port, wherein the gas layer above the gas outlet port is warmer than the gas layer below the gas outlet port.

20. The apparatus of claim 1, further comprising at least two gas layers having distinct temperature profiles wherein a first gas layer is above a second gas layer and the border between the first and second gas layers is near the level of the gas outlet port, wherein the gas layer above the gas outlet port is warmer than the gas layer below the gas outlet port.

21. The apparatus of claim 1, wherein the collection reservoir is joined to the spray chamber by an adapter.

22. The apparatus of claim 1, wherein the collection reservoir is joined to the spray chamber by a flange.

23. The apparatus of claim 1, wherein the collection reservoir is joined to the spray chamber by a removable flange

24. The apparatus of claim 1, wherein the collection reservoir is an integral part of the spray chamber.

25. The apparatus of claim 1 further comprising an outlet port leading from the collection reservoir for removing collection fluid.

26. The apparatus of claim 1 further comprising an outlet port leading from the collection reservoir for removing collection fluid and particles.

27. A method for preparing solid particles comprising,
forming in an enclosed container a first gaseous temperature zone having a temperature above the freezing or precipitation points of a particle forming solution,
forming a pool of a cold collection fluid in the closed container,
preparing a particle-forming mixture of a particle-forming material in a liquid,
spraying the mixture through the first gaseous temperature zone directly into the pool of collection fluid and forming a solid particle.

28. The method for preparing a solid particle of claim 27, further comprising forming a second gaseous temperature zone below the first gaseous temperature zone.

29. The method for preparing a solid particle of claim 27, further comprising forming a second gaseous temperature zone below the first gaseous temperature zone wherein at least a portion of the second gaseous temperature zone has a temperature below a temperature in the first temperature zone.

30. The method for preparing a solid particle of claim 27, wherein the solid particle is extracted with an anti-solvent to remove at least a portion of the solvent.

31. The method for preparing a solid particle of claim 27, wherein the solid particle is extracted with an anti-solvent to remove at least a portion of the solvent at a temperature below the freezing or precipitation point of the mixture of a particle-forming material in the liquid.

32. The method for preparing a solid particle of claim 27, wherein the pool of cold collection fluid is formed and the solid particle is extracted in the same vessel.

33. The method for preparing a solid particle of claim 27, wherein the pool of cold collection fluid is formed and the solid particle is extracted and dried in the enclosed container.

34. The method for preparing a solid particle of claim 27, wherein the cold collection fluid is a liquefied gas.

35. The method for preparing a solid particle of claim 27, wherein the cold collection fluid is an anti-solvent.

36. The method for preparing a solid particle of claim 27, wherein the step of preparing a particle-forming mixture of a particle-forming material in a liquid further comprises adding an active agent.

37. The method for preparing a solid particle of claim 27, further comprising forming a second gaseous temperature zone below the first gaseous temperature zone wherein at least a portion of the second gaseous temperature zone has a temperature below the freezing or precipitation temperature of the particle-forming mixture.

38. The method for preparing a solid particle of claim 27, wherein the step of spraying the particle-forming mixture through the first gaseous temperature zone directly into the pool of collection fluid to form a frozen particle in the enclosed container further comprises evaporating a portion of the solvent from the liquid spray droplets in the first gaseous temperature zone.

39. The method for preparing a particle of claim 27, wherein the collection fluid is an antisolvent and further comprising selecting a particle-forming material solvent and antisolvent such that the Stefan number is in a range such that a particle having a solid core can form and forming a particle having a solid core.

40. The method for preparing a particle of claim 27, wherein the collection fluid is an antisolvent and further comprising selecting a particle-forming material solvent and antisolvent such that the Stefan number is in a range such that a particle having a phase-separated structure can form and forming a particle having a phase-separated structure.

41. The method for preparing a particle of claim 27, wherein the particle-forming material includes PLGA and the collection fluid is an antisolvent and further comprising selecting a particle-forming material solvent and antisolvent such that the Stefan number is greater than about 1 and forming a particle having a solid core.

42. A method for forming an amorphous dispersion of a compound in a solid particle comprising, dissolving a compound and a particle-forming material in a solvent, atomizing the solution into an inert cryogenic liquid, freezing the particles, removing a portion of the cryogenic liquid, extracting a portion of the solvent from the particle into a non-solvent and isolating solid particles containing an amorphous dispersion of the compound.

43. The method of forming an amorphous dispersion of claim 42, wherein the compound is a poorly aqueous soluble molecule.

44. A method for forming an amorphous dispersion of a compound in a solid particle comprising, dissolving a compound and a particle-forming material in a solvent, atomizing the solution into a cryogenic non-solvent, freezing the particles, extracting a portion of the solvent from the particle into the non-solvent and isolating solid particles containing an amorphous dispersion of the compound.

45. The method of forming an amorphous dispersion of claim 44, wherein the compound is a poorly aqueous soluble molecule.

46. The method of forming an amorphous dispersion of claim 44, wherein the temperature of the cryogenic non-solvent is increased during the solvent extraction step.
